(19) United States
(12) Patent Application Publication
(10) Pub. No.: US 2017/0248579 A1
(43) Pub. Date: Aug. 31, 2017

(54) AMYOTROPHIC LATERAL SCLEROSIS (ALS) BIOMARKERS AND USES THEREOF

(71) Applicants: The UAB Research Foundation, Birmingham, AL (US); THE UNITED STATES GOVERNMENT AS REPRESENTED BY THE DEPARTMENT OF VETERANS AFFAIRS, Washington, DC (US)

(72) Inventor: Peter H. King, Birmingham, AL (US)

(73) Assignee: The UAB Research Foundation, Birmingham, AL (US)

(21) Appl. No.: 15/510,490

(22) PCT Filed: Sep. 10, 2015
(86) PCT No.: PCT/US2015/049440

§ 371 Date: Mar. 10, 2017

Related U.S. Application Data
(60) Provisional application No. 62/048,554, filed on Sep. 10, 2014.

(51) Int. Cl. G01N 33/50; G01N 33/68

(52) U.S. Cl. CPC G01N 33/5023; G01N 33/6809; G01N 33/6896; G01N 2800/28; G01N 2800/2835

(57) ABSTRACT

Provided herein are Amyotrophic lateral sclerosis (ALS) biomarkers and methods of using these ALS biomarkers to diagnose and treat ALS.

This application claims the benefit of U.S. Provisional Application No. 62/048,554, filed Sep. 10, 2014, which is hereby incorporated herein in its entirety.

STATEMENT REGARDING FEDERALLY FUNDED RESEARCH

This invention was made with government support under grant numbers NS064133, R21NS081743, R21NS085497 and NS057664 awarded by the National Institute of Neurological Disorders; and Merit Review Award No. BX001148, awarded by the United States Department of Veterans Affairs Biomedical Laboratory Research and Development Program. The government has certain rights in this invention.

### BACKGROUND

Amyotrophic lateral sclerosis (ALS) is a devastating and fatal neurodegenerative disease with no definite etiology identified and no effective treatment. Therefore, methods of diagnosing and treating ALS are needed.

### SUMMARY

Provided herein is a method for diagnosing amyotrophic lateral sclerosis (ALS) in a subject comprising isolating a sample (e.g., a muscle biopsy or blood sample) from the subject and detecting the level of SMAD1, SMAD2, SMAD5, and/or SMAD8 in the sample, an increase in SMAD1, SMAD2, SMAD5, and/or SMAD8, as compared to a control, indicating the subject has or is at risk for developing ALS.

Further provided is a method of treating a subject with or at risk of developing ALS. The method includes isolating a sample from the subject with ALS or at risk of developing ALS; detecting the level of SMAD1, SMAD2, SMAD5, and/or SMAD8 in the sample, an increase in SMAD1, SMAD2, SMAD5, and/or SMAD8, as compared to a control, indicating the subject has or is at risk for developing ALS; and administering an effective amount of an agent that decreases the level of SMAD1, SMAD2, SMAD5, and/or SMAD8 in the subject. Also provided is a method for determining the efficacy of a selected treatment for ALS in a subject. The steps of the method include (a) isolating a first sample from the subject before the selected treatment; (b) detecting a first level of SMAD1, SMAD2, SMAD5, and/or SMAD8 in the first sample; (c) treating the subject with the selected treatment; d) isolating a second sample from the subject after the selected treatment; (e) detecting a second level of SMAD1, SMAD2, SMAD5, and/or SMAD8 in the second sample of step (d); (f) comparing the first and second levels of SMAD1, SMAD2, SMAD5, and/or SMAD8 detected in step (b) and (e), a decrease in the second level of SMAD1, SMAD2, SMAD5, and/or SMAD8 detected in step (e) indicating that the selected treatment is effective for treating ALS in the subject. The selected treatment is adjusted accordingly, if the second level is not decreased.

Also provided herein is a method for diagnosing amyotrophic lateral sclerosis (ALS) in a subject. The method includes isolating a sample from the subject and detecting the level of TGFβ1, TGFβ2, TGFβ3 and/or BMP4 in the sample. An increase in TGFβ1, TGFβ2, TGFβ3 and/or BMP4, as compared to a control, indicates the subject has or is at risk for developing ALS.

Further provided is a method of treating a subject with or at risk of developing ALS. The method comprises isolating a sample from the subject with ALS or at risk of developing ALS; detecting the level of TGFβ1, TGFβ2, TGFβ3 and/or BMP4 in the sample, an increase in TGFβ1, TGFβ2, TGFβ3 and/or BMP4, as compared to a control, indicating the subject has or is at risk for developing ALS; and administering an effective amount of an agent that decreases the level of TGFβ1, TGFβ2, TGFβ3 and/or BMP4 in the subject.

Also provided is a method for determining the efficacy of a selected treatment for ALS in a subject. The method includes the steps of (a) isolating a first sample from the subject before the selected treatment; (b) detecting the first level of TGFβ1, TGFβ2, TGFβ3 and/or BMP4 in the first sample; (c) treating the subject with the selected treatment; (d) isolating a second sample from the subject after the selected treatment; (e) detecting a second level of TGFβ1, TGFβ2, TGFβ3 and/or BMP4 in the sample of step (d); (f) comparing the first and second levels of TGFβ1, TGFβ2, TGFβ3 and/or BMP4 detected in step (b) and (e), a decrease in the level of TGFβ1, TGFβ2, TGFβ3 and/or BMP4 detected in step (e) indicating that the selected treatment is effective for treating ALS in the subject. The selected treatment is adjusted accordingly, if the second level is not decreased.

Also provided herein is a method for diagnosing amyotrophic lateral sclerosis (ALS) in a subject. The method includes isolating a sample from the subject and detecting the level of SMAD1, SMAD2, SMAD5, SMAD8, TGFI31, TGFI32, TGFI33 and/or BMP4 in the sample. An increase in SMAD1, SMAD2, SMAD5, SMAD8,TGFβ1, TGFβ2, TGFβ3 and/or BMP4, as compared to a control, indicates the subject has or is at risk for developing ALS.

Further provided is a method of treating a subject with or at risk of developing ALS. The method comprises isolating a sample from the subject with ALS or at risk of developing ALS; detecting the level of SMAD1, SMAD2, SMAD5, SMAD8, TGFβ1, TGFβ2, TGFβ3 and/or BMP4 in the sample, an increase in SMAD1, SMAD2, SMAD5, SMAD8, TGFβ1, TGFβ2, TGFI33 and/or BMP4, as compared to a control, indicating the subject has or is at risk for developing ALS; and administering an effective amount of an agent that decreases the level of SMAD1, SMAD2, SMAD5, SMAD8, TGFβ1, TGFβ2, TGFβ3 and/or BMP4 in the subject.

Also provided is a method for determining the efficacy of a selected treatment for ALS in a subject. The method includes the steps of (a) isolating a first sample from the subject before the selected treatment; (b) detecting the first level of SMAD1, SMAD2, SMAD5, SMAD8, TGFβ1, TGFβ2, TGFβ3 and/or BMP4 in the first sample; (c) treating the subject with the selected treatment; (d) isolating a second sample from the subject after the selected treatment; (e) detecting a second level of SMAD1, SMAD2, SMAD5, SMAD8, TGFβ1, TGFβ2, TGFβ3 and/or BMP4 in the sample of step (d); (f) comparing the first and second levels of SMAD1, SMAD2, SMAD5, SMAD8, TGFβ1, TGFβ2, TGFβ3 and/or BMP4 detected in step (b) and (e), a decrease in the level of SMAD1, SMAD2, SMAD5, SMAD8, TGFβ1, TGFβ2, TGFβ3 and/or BMP4 detected in step (e) indicating that the selected treatment is effective for treating ALS in the subject. The selected treatment is adjusted accordingly, if the second level is not decreased.

### DESCRIPTION OF DRAWINGS

FIG. 1 shows that Smad1, Smad5, and Smad8 mRNA expression is upregulated in muscle samples from ALS patients. FIG. 1A shows the results when total RNA from muscle biopsy samples was analyzed by qRT-PCR for Smad1,5 and 8 mRNA expression in patients with ALS (n=27), myopathy (n=11), neuropathy (n=9) or no neuromuscular disease (n=13). RQ, relative quantity. *, P<0.05; ****, P <0.0001. FIG. 1B shows the correlation plots between the Smad cycle threshold

(Ct) values from the ALS muscle samples.

FIG. 2 shows that Smad1,5 and 8 mRNAs are upregulated in muscle samples from a mouse model of ALS (G93A SOD1) and increase further with disease progression. FIG. 2A shows the results of rotarod testing and weight measurement of G93A SOD1 mice over the course of the disease. Gastrocnemius muscle samples were obtained at the ages shown and also at day 40. Control muscle samples were obtained from age-matched non-transgenic littermates. FIG. 2B shows Smad1, FIG. 2C shows Smad5, and FIG. 2D shows Smad8 mRNA levels quantitated using qRT-PCR and normalized to an internal housekeeping (GAPDH) control. All data points represent the mean±SEM of 3-8 independent mice. *, P<0.01; **, P<0.001.

FIG. 3 shows that Smad1, 5, 8 mRNAs are not elevated in G93A SOD1 mouse spinal cord or brain tissues. Spinal cord and brain tissues from G93A SOD1 and non-transgenic littermates (WT) were assessed by qRT-PCR for Smad1, 5, and 8 expression at the ages indicated. Values were expressed relative to WT at day 60 which was set at 1. Data represent the mean±SEM of 3 mice.

FIG. 4 shows that Smad1, 5, 8 protein expression and activation increase with disease progression in the G93A SOD1 mouse. FIG. 4A shows a representative Western blot of gastrocnemius samples from G93A SOD1 mice (M) and littermate controls (W) at different ages. Antibodies are shown to the right of the blot. FIG. 4B shows total Smad1,5,8 quantified by densitometry, normalized to GAPDH, and compared to non-transgenic littermate controls. FIG. 4C shows p-Smad/t-Smad ratios calculated in G93A mice and compared to littermate controls (set at 1). Data points represent the mean±SEM of 4-6 mice in each group. *, P<0.05; ****P<0.0001.

FIG. 5 shows that Smad1,5,8 is activated in mouse and human ALS muscle samples. Muscle sections from G93A SOD1 mouse (day 60) and a biopsy from a patient with ALS were stained with p-Smad1,5,8 antibody, WGA lectin, and Hoechst as indicated. For the G93A SOD1 mouse, the bottom panels represent higher power views of areas highlighted by asterisks. The arrows indicate co-localization of p-Smad1,5,8 with Hoechst (nuclear) signal, and arrowheads show co-localization with WGA. An age-matched non-transgenic littermate and a normal human muscle biopsy sample were used as controls. Scale bars, 50 μM.

FIG. 6 shows that p-Smad 1,5,8 accumulates as the disease progresses. Increased p-Smad1,5,8 staining at later stage of disease in ALS mouse muscle (131 d) and in a patient with end-stage ALS. Scale bars, 50 μM.

FIG. 7 shows that muscle Smads are induced and activated in a peripheral nerve injury model. FIG. 7A shows Basso mouse scale for locomotion (BMS) 2 days post nerve injury (PNI) and the Von Frey mechanical sensitivity test 1 week post nerve injury (PNI) (except for the 2.5 d group). Mice were subjected to sciatic nerve injury (SNI) as described in the Examples. To determine efficacy of the injury, mice were assessed behaviorally with the BMS and Von Frey tests. There was a significant reduction in locomotion and mechanical threshold to withdraw, indicative of nerve injury.***, P<0.001. FIG. 7B shows Smad mRNA levels assessed in the gastrocnemius muscle by qRT-PCR at 2.5 d,1 week and 4 weeks post nerve injury (PNI) and compared to a control group that underwent sham surgery. All RNA values were expressed as a fold-change over the 1 week sham control group. Data are from 3-6 mice in each group. FIG. 7C is a representative Western blot of muscle protein extracts from injured (I) or sham controls (S). Antibodies are shown to the left of the blots. FIG. 7D shows quantitative densitometry of three Western blots representative of 3-6 mice in each test group. For t- and p-Smad quantitation, band densities were adjusted to the loading control (GAPDH) and then expressed as a fold-change over the sham control at one week PNI. P-Smad/t-Smad ratios were calculated and expressed as a fold-change over the sham control group at week 1 PNI. *, P<0.05; **, P<0.005.

FIG. 8 shows that elevation of Smad mRNA is delayed in the forelimb muscles of ALS mice. Forelimb muscles from G93A and WT mice were assessed by qRT-PCR for Smad mRNA expression. All data points represent the mean±SEM of 3 mice. *, P<0.05.

FIG. 9 shows that upregulation of p-Smad is delayed in the forelimb muscles of ALS mice. Western blot analysis of forelimb muscle tissue using phosphorylated and total-Smad1,5,8 antibodies at the ages shown. This blot is representative of two blots done on two independent G93A and WT mice with similar results.

FIG. 10 shows that TGF-β mRNA is increased in muscle samples from ALS patients. FIG. 16A shows total RNA from muscle biopsy samples analyzed by qRT-PCR for TGF-β1, 2, and 3 mRNA expression in patients with ALS (n=27), myopathy (n=11), neuropathy (n=9) or no neuromuscular disease (n=13). RQ, relative quantity. *, p<0.05; **,<0.005; ***<0.0005; ****<0.0001. FIG. 10B shows the correlation of TGF-β isoform mRNA levels (expressed as the Ct value from qRT-PCR). FIG. 10C shows the correlation between muscle grade of biopsied ALS muscle samples (as measured by the Medical Research Council scale) and TGF-β mRNA.

FIGS. 11 shows that TGF-βand Smad mRNA levels correlate in ALS muscle samples. Smad1, 5 and 8 mRNA levels were determined by qRT-PCR and compared with TGF-β mRNA levels from the same ALS muscle biopsy sample (Ct values are shown).

FIG. 12 shows that TGF-β mRNA levels are increased at early stages of ALS in the G93A SOD1 mouse. Total RNA was isolated from G93A SOD1 mice and littermate controls (WT) at 40, 60 and 105 d (preclinical stages as described in Si et al., “Smads as muscle biomarkers in amyotrophic lateral sclerosis” Ann Clin Trans' Neurol. 1(10):778-87 (2014))), and early and late clinical stages (125 and 150 d). Samples were analyzed by qRT-PCR for TGF-β1, 2, 3 and BMP4 mRNA expression.

Data points represent the mean ±SE of 6-8 mice. *p<0.05; **<0.005; ****<0.0001. RQ, relative quantity.

FIG. 13 shows that TGF-β protein is elevated in G93A mouse muscle. FIG. 13A shows muscle lysates from G93A SOD1 mice (M) and non-transgenic littermates (W) that were assessed for TGF-β1 by ELISA and Western blot. ELISA values were determined by comparison to a standard curve. A representative Western blot of the lysates (under reducing conditions) shows expression of mature TGF-β1. ELISA data represent the mean±SE of 3 mice. The Western blot was repeated once with the similar results. FIG. 13B shows confocal photomicrographs of G93A or WT muscle sections using a TGF-β1 antibody. WGA, wheat germ agglutinin. Size marker=50 microns. FIG. 13C is a representative Western blot of TGF-β2 and P3 in mouse muscle showing increased levels of the unprocessed peptide in mutant versus control samples. Quantitative densitometry of TGF-β ligands was performed on three Western blots from three independent mouse samples. Data are shown as fold-increase over WT controls. *p<0.05 for each age.

FIG. 14 shows that TGF-β1 is increased in human ALS muscle. FIG. 14A shows acid activated protein lysates from human muscle biopsy samples that were assessed for TGF-β1 by ELISA and compared to a standard curve. Data points are the mean±SE. Samples include ALS (n=12); neuropathy (n=7); myopathy (n=8); normal (n=4). *, p<0.05. FIG. 14B shows confocal photomicrographs of ALS and control muscle samples labeled with an anti-TGF-β1 antibody. Bx, biopsy specimen; Aut, autopsy specimen; Ctl, normal biopsy. Size marker=50 microns.

FIG. 15 shows that TGF-β induces Smads1, 5 and 8 in cultured C2C12 muscle cells. FIG. 15A shows data obtained from C2C12 muscle cells that were treated with TGF-β ligands for the time frames shown and then assessed for Smad1, 5, and 8 mRNA expression by qRT-PCR. Data points were expressed as a fold-increase over vehicle treated cells and represent the mean±SE of 6-8 independent samples. ** P<0.005; ***<0.0005; ****<0.0001. FIG. 15B shows data obtained from C2C12 cells that were treated with TGF-βligands for the time frames shown and assessed for p- and t-Smad1, 5, 8 by Western blot. The experiment was repeated one time with similar results.

FIG. 16 shows that Smad2 and 3 are increased in ALS muscle. FIG. 16A shows total RNA from human muscle biopsy samples that were analyzed by qRT-PCR for Smad2 and 3 mRNA expression as described in FIG. 1. FIG. 16B shows Smad2 and3 mRNA levels in the G93A mouse that were determined by qRT-PCR. Data points represent the mean±SE of 3-4 mice. FIG. 16C is a Western blot of C2C12 cells treated with TGF-βligands for the times shown. Antibodies are shown to the left. RQ, relative quantity. *, P<0.05; **<0.005; ****<0.0001.

### DESCRIPTION

ALS is a devastating and fatal neurodegenerative disease with no definite etiology identified and no current effective treatment. The hallmark of ALS is progressive muscle atrophy and weakness, leading to loss of limb and bulbar function. While degeneration of motor neurons underlies these clinicopathological changes, skeletal muscle plays an active role in disease progression.

Provided herein is a method for diagnosing amyotrophic lateral sclerosis (ALS) in a subject. The method includes isolating a sample from the subject and detecting the level of mothers against decapentaplegic homolog 1 (SMAD1), mothers against decapentaplegic homolog 2 (SMAD2), mothers against decapentaplegic homolog 5 (SMAD5), and/or mothers against decapentaplegic homolog 8 (SMAD8) in the sample. An increase in SMAD1, SMAD2, SMAD5, and/or SMAD8, as compared to a control, indicates the subject has or is at risk for developing ALS.

Also provided is a method for diagnosing amyotrophic lateral sclerosis (ALS) in a subject comprising isolating a sample from the subject and detecting the level of phosphorylation of SMAD1, SMAD2, SMAD5 and/or SMAD8. An increase in the level of phosphorylation of SMAD1, SMAD2, SMAD5, and/or SMAD8, as compared to a control, indicates the subject has or is at risk for developing ALS.

Also provided is a method for diagnosing amyotrophic lateral sclerosis (ALS) in a subject comprising isolating a sample from the subject and detecting the ratio of phosphorylated SMAD to total SMAD (p-SMAD/t-SMAD) An increase in the p-SMAD/t-SMAD ratio, as compared to a control, indicates the subject has or is at risk for developing ALS.

Also provided is a method for diagnosing amyotrophic lateral sclerosis (ALS) in a subject comprising isolating a sample from the subject and detecting the level of transforming growth factor beta 1 (TGFβ1), transforming growth factor beta 2 (TGFβ2), transforming growth factor beta 3 (TGFβ3) and/or bone morphogenetic protein 4 (BMP4) in the sample. An increase in TGFβ1, TGFβ2, TGFβ3 and/or BMP4, as compared to a control, indicates the subject has or is at risk for developing ALS.

Further provided is a method for diagnosing amyotrophic lateral sclerosis (ALS) in a subject. The method includes isolating a sample from the subject and detecting the level of SMAD1, SMAD2, SMAD5, SMAD8, TGFβ1, TGFβ2, TGFβ3 and/or BMP4 in the sample. An increase in SMAD1, SMAD2, SMAD5, SMAD8, TGFβ1, TGFβ2, TGFβ3 and/or BMP4, as compared to a control, indicates the subject has or is at risk for developing ALS.

In the methods provided herein, nucleic acids (e.g., mRNA) encoding SMAD1, SMAD2, SMAD5, SMAD8, TGFβ1, TGFβ2, TGFβ3 and/or BMP4 or fragments thereof can be detected. Alternatively or additionally, SMAD1, SMAD2, SMAD5, SMAD8 TGFβ1, TGFβ2, TGFβ3 and/or BMP4 proteins or fragments thereof can be detected. Examples of mRNAs encoding human SMAD1 can be found under GenBank Accession Nos. NM_001003688.1 and NM_005900.2. Examples of amino acid sequences for human SMAD1 can be found under GenBank Accession Nos. NP_001003688.1 and NP_005891.1. Examples of mRNAs encoding human SMAD2 can be found under GenBank Accession Nos. NM_001003652.3, NM_001135937.2 and NM_005901.5. Examples of amino acid sequences for human SMAD2 can be found under GenBank Accession Nos. NP_001003652.1, NP_001129409.1 and NP_005892.1. Examples of mRNAs encoding human SMAD5 can be found under GenBank Accession Nos. NM_001001419.2, NM_001001420.2 and NM_005903.6 and examples of amino acid sequences for human SMAD5 can be found under GenBank Accession Nos. NP_001001419.1, NP_001001420.1 and NP_005894.3. Examples of mRNAs encoding human SMAD8 can be found under GenBank Accession Nos. NM_001127217.2, and NM_005905.5 and examples of amino acid sequences for human SMAD8 can be found under GenBank Accession Nos. NP_001120689.1, and NP_005896.1. An example of an mRNA encoding human TGFβ1 can be found under GenBank Accession No. NM_000660.5. An example of an amino acid sequence for human TGFβ1 can be found under GenBank Accession No. NP_000651.3. Examples of mRNAs encoding human TGFβ2 can be found under GenBank Accession Nos. NM_001135599.2 and NM_003238.3. Examples of amino acid sequences for human TGFβ2 can be found under GenBank Accession Nos. NP_001129071.1 and NP_003229.1. An example of an mRNA encoding human TGFβ3 can be found under GenBank Accession No. NM_003239.3.An example of an amino acid sequence for human TGFβ3 can be found under GenBank Accession No. NP_003230.1. Examples of mRNAs encoding human BMP4 can be found under GenBank Accession Nos. NM_001202.3 and NM_130850.2. Examples of amino acid sequences for human BMP4 can be found under GenBank Accession Nos. NP_001193.2 and NP_570911.2.

The amount of a mRNA encoding SMAD1, SMAD2, SMAD5, SMAD8 TGFβ1, TGFβ2, TGFβ3 and/or BMP4 in a cell or in a sample can be determined by methods standard in the art for quantitating nucleic acids such as in situ hybridization, quantitative PCR, RT-PCR, Taqman assay, Northern blotting, ELISPOT, dot blotting, etc., as well as any other method now known or later developed for quantitating the amount of a nucleic acid in a cell or released from a cell. The amount of SMAD1, SMAD2, SMAD5, SMAD8, TGFβ1, TGFβ2, TGFβ3 and/or BMP4 protein or a fragment thereof in a cell or in a sample, can be determined by methods standard in the art for quantitating proteins such as densitometry, absorbance assays, fluorometric assays, Western blotting, ELISA, radioimmunoassay, ELISPOT, immunoprecipitation, immunofluorescence (e.g., FACS), immunohistochemistry, etc., as well as any other method now known or later developed for quantitating a specific protein in or produced by a cell. Imaging techniques can be used to detect SMAD1, SMAD2, SMAD5, SMAD8, TGFβ1, TGFβ2, TGFβ3 and/or BMP4. For example imaging techniques that use target-specific contrast agents can be used to detect SMAD1, SMAD2, SMAD5, SMAD8, TGFβ1, TGFβ2, TGFβ3 and/or BMP4 protein in a sample or in a subject. In another example, an imaging agent, such as a labeled binding protein, antibody or a functional fragment thereof that specifically binds to SMAD1, SMAD2, SMAD5, SMAD8, TGFβ1, TGFβ2, TGFβ3 or BMP4 can be administering to a subject. The imaging agent is administered in an amount effective for diagnostic use in a mammal such as a human. The localization and accumulation of the imaging agent is then detected after it has bound to SMAD1, SMAD2, SMAD5, SMAD8, TGFβ1, TGFβ2, TGFβ3 or BMP4 present in the sample or the subject. The localization and accumulation of the imaging agent can be detected by radionucleide imaging, radioscintigraphy, nuclear magnetic resonance imaging, computed tomography, positron emission tomography, computerized axial tomography, X-ray or magnetic resonance imaging method, fluorescence detection and/or chemiluminescent detection. The imaging agent can be labeled with a radionuclide or a non-radioactive indicator.

Methods for detecting protein phosphorylation are known in the art and include, but are not limited to, ELISA, radioimmunassay, Western blot and mass spectrometry, to name a few. As used herein, p-SMAD can be the amount of one or more phosphorylated SMADs, for example, p-SMAD1, pSMAD2, p-SMAD5 and/or p-SMAD8. One or more antibodies that specifically bind to the phosphorylated form of SMAD1, SMAD2, SMAD5 or SMAD8 protein can be used to detect the level of phosphorylated SMAD. Antibodies that bind to the phosphorylated and unphosphorylated form of SMAD1, 2, 5 and 8 can be used to detect the total amount of SMAD1,2,5,8 in a sample. Optionally, antibodies that bind to the phosphorylated and unphosphorylated form of SMAD1, 5 and 8 can be used to detect the total amount of SMAD1,5,8 in a sample. For example, and not to be limiting, the p-SMAD/t-SMAD ratio can be the ratio of p-SMAD1,2,5,8/t-SMAD1,2,5,8 or the ratio of p-SMAD1,5,8/t-SMAD1,5,8. It is understood that the phosphorylated form of a SMAD is also an activated form of a SMAD. Therefore, the amount of one or more phosphorylated SMADs or the level of phosphorylation in a sample is also the amount of activated SMADs or the level of activation in a sample.

Microarray technology can also be used to detect differential expression of SMAD1, SMAD2, SMAD5, SMAD8, TGFβ1, TGFβ2, TGFβ3 and/or BMP4 in a biological sample from a subject and a control sample. In this method, polynucleotide sequences of interest are plated, or arrayed, on a microchip substrate, for example, sequences that specifically hybridize to nucleic acid sequences encoding SMAD1, SMAD2, SMAD5, SMAD8, TGFβ1, TGFβ2, TGFβ3 and/or BMP4. The arrayed sequences are contacted with nucleic acids obtained from the subject's biological sample or from a control sample under appropriate hybridization conditions to determine the level of SMAD1, SMAD2, SMAD5, SMAD8, TGFβ1, TGFβ2, TGFβ3 and/or BMP4 in the biological sample as compared to a control sample.

As used throughout, a control can be, for example, a subject that does not have ALS, or a reference amount or reference value of SMAD1, SMAD2, SMAD5, SMAD8 TGFβ1, TGFβ2, TGFβ3 and/or BMP4 that is indicative of a subject that does not have ALS. Optionally, the control is a level of SMAD1, SMAD2, SMAD5, SMAD8 TGFβ1, TGFβ2, TGFβ3 and/or BMP4 that is higher than the level of SMAD1, SMAD2, SMAD5, SMAD8 TGFβ1, TGFβ2, TGFβ3 and/or BMP4 in a subject that does not have ALS and a detected level comparable to or higher than the control level indicates that the subject has or is at risk of developing ALS. Optionally, the methods further comprise selecting a subject having or suspected of having ALS.

A control level can be obtained from a control sample, which can comprise either a sample obtained from a control subject (e.g., from the same subject at a different time than the biological sample), or from a second subject, or can comprise a known standard. Optionally, the control level is taken from an individual of a population having no signs of ALS for an individual of that population. For example, the control level may be the level of SMAD1, SMAD2, SMAD5, SMAD8 TGFβ1, TGFβ2, TGFβ3 and/or BMP4 obtained from a twenty-five year old male or female having no particular signs or symptoms of ALS or an age matched co-hort.

As used throughout, an increase can be an increase of about 10%, 20%, 30%, 40%, 50%, 60%, 70%, 80%, 90%, 100%, 200%, 300%, 400% or greater as compared to a control sample or a control value. An increase can also be a 1.1 fold, 1.2 fold, 1.3 fold, 1.4 fold, 1.5 fold, 2-fold, 3-fold, 4-fold, 5-fold, 10-fold, 15-fold, 20-fold, 25-fold increase or greater as compared to a control sample or a control value. In the methods provided herein, a decrease can be about 5%, 10%, 20%, 30%, 40%, 50%, 60%, 70%, 80%, 90%, 100% or any percentage in between as compared to a control sample or a control value.

As used throughout, by subject is meant an individual. Preferably, the subject is a mammal such as a primate, and, more preferably, a human. Non-human primates are subjects as well. The term subject includes domesticated animals (such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

In the methods set forth herein, SMAD1, SMAD2, SMAD5, SMAD8, TGFβ1, TGFβ2, TGFβ3 and/or BMP4 can be detected in a biological sample derived from a subject, and, more particularly, the sample can include, but is not limited to, a cell, tissue or biological fluid from the subject. For example, the sample can be a tissue biopsy (for example, a needle biopsy), blood, plasma, serum, bone marrow, cerebrospinal fluid, urine, saliva, muscle biopsy (for example, skeletal muscle), tissue infiltrate and the like. Optionally, a muscle biopsy can be obtained from a hindlimb or lower extremity of the subject. Optionally the biological sample includes cells derived from the subject and cell culture medium.

Also provided herein is a method for determining the progression of ALS in a subject. The method includes (a) isolating a first sample from the subject with ALS; (b) detecting a first level of SMAD1, SMAD2, SMAD5, and/or SMAD8 in the sample; (c) isolating a second sample from the subject at a later time point; (d) detecting a second level of SMAD1, SMAD2, SMAD5, and/or SMAD8 in the sample of step (d); e) comparing the first and second levels of SMAD1, SMAD2, SMAD5, and/or SMAD8 detected in step b) and d). An increase in the second level of SMAD1, SMAD2, SMAD5, and/or SMAD8 detected in step (d) indicates that the ALS has progressed in the subject, and a decrease in the second level of SMAD1, SMAD2, SMAD5, and/or SMAD8 detected in step (d) indicates that ALS has improved in the subject. No difference between the first and second levels of SMAD1, SMAD2, SMAD5, and/or SMAD8 detected in step b) and d) indicates that ALS has not progressed in the subject.

Also provided herein is a method for determining the progression of ALS in a subject. The method includes (a) isolating a first sample from the subject with ALS; (b) detecting a first level of phosphorylated SMAD1, SMAD2, SMAD5 and/or SMAD8 in the sample; (c) isolating a second sample from the subject at a later time point; (d) detecting a second level of phosphorylated SMAD1, SMAD2, SMAD5 and/or SMAD8 in the sample of step (d); e) comparing the first and second levels of phosphorylated SMAD1, SMAD2, SMAD5 and/or SMAD8 detected in step b) and d). An increase in the second level of phosphorylated SMAD1, SMAD2, SMAD5 and/or SMAD8 detected in step (d) indicates that the ALS has progressed in the subject, and a decrease in the second level of phosphorylated SMAD1, SMAD2, SMAD5 and/or SMAD8 detected in step (d) indicates that ALS has improved in the subject. No difference between the first and second levels of phosphorylated SMAD1, SMAD2, SMAD5 and/or SMAD8 detected in step b) and d) indicates that ALS has not progressed in the subject.

Also provided herein is a method for determining the progression of ALS in a subject. The method includes (a) isolating a first sample from the subject with ALS; (b) detecting a first p-SMAD/t-SMAD ratio in the sample; (c) isolating a second sample from the subject at a later time point; (d) detecting a second p-SMAD/t-SMAD) ratio in the sample of step (d); e) comparing the first and second p-SMAD/t-SMAD ratio detected in step b) and d). An increase in the second p-SMAD/t-SMAD ratio detected in step (d) indicates that the ALS has progressed in the subject, and a decrease in the second p-SMAD/t-SMAD ratio detected in step (d) indicates that ALS has improved in the subject. No difference between the first and second p-SMAD/t-SMAD ratio detected in step b) and d) indicates that ALS has not progressed in the subject.

Also provided is a method for determining the progression of ALS in a subject that includes the steps of (a) isolating a first sample from the subject with ALS; (b) detecting a first level of TGFβ1, TGFβ2, TGFβ3 and/or BMP4 in the sample; (c) isolating a second sample from the subject at a later time point; (d) detecting a second level of TGFβ1, TGFβ2, TGFβ3 and/or BMP4 in the sample of step (d); (e) comparing the first and second levels of TGFβ1, TGFβ2, TGFβ3 and/or BMP4 detected in step (b) and (d), an increase in the second level of TGFβ1, TGFβ2, TGFβ3 and/or BMP4 detected in step (d) indicating that ALS has progressed in the subject; a decrease in the second level of TGFβ1, TGFβ2, TGFβ3 and/or BMP4 detected in step (d) indicating that ALS has improved in the subject; and no difference between the first and second levels of TGFβ1, TGFβ2, TGFβ3 and/or BMP4 detected in step (b) and (d) indicating that ALS has not progressed in the subject. BMP4 is elevated particularly at late stages of disease progression.

Also provided is a method for determining the progression of ALS in a subject that includes the steps of (a) isolating a first sample from the subject with ALS; (b) detecting a first level of SMAD1, SMAD2, SMAD5, SMAD8, TGFβ1, TGFβ2, TGFβ3 and/or BMP4 in the sample; (c) isolating a second sample from the subject at a later time point; (d) detecting a second level of SMAD1, SMAD2, SMAD5, SMAD8, TGFβ1, TGFβ2, TGFβ3 and/or BMP4 in the sample of step (d); (e) comparing the first and second levels of SMAD1, SMAD2, SMAD5, SMAD8, TGFβ1, TGFβ2, TGFβ3 and/or BMP4 detected in step (b) and (d), an increase in the second level of SMAD1, SMAD2, SMAD5, SMAD8, TGFβ1, TGFβ2, TGFβ3 and/or BMP4 detected in step (d) indicating that ALS has progressed in the subject; a decrease in the second level of SMAD1, SMAD2, SMAD5, SMAD8, TGFβ1, TGFβ2, TGFβ3 and/or BMP4 detected in step (d) indicating that ALS has improved in the subject; and no difference between the first and second levels of SMAD1, SMAD2, SMAD5, SMAD8, TGFβ1, TGFβ2, TGFβ3 and/or BMP4 detected in step (b) and (d) indicating that ALS has not progressed in the subject. BMP4 is elevated, particularly at late stages of disease progression.

In the methods for determining progression of ALS, a sample can be obtained from the subject any time after diagnosis of ALS. A sample can then be obtained at a later time point, for example, one, two, three, four, five, six, seven, eight, nine, ten, eleven, twelve months later, two years later or three years later to determine if the disease has progressed. These time points are merely exemplary, as the second sample can be obtained from the subject at any time after obtaining the first sample from the subject.

The methods set forth herein can also be used for staging ALS. For example, SMAD1, SMAD2, SMAD5, SMAD8, TGFβ1, TGFβ2, TGFβ3 and/or BMP4 can be detected in a biological sample from a subject. The expression level of SMAD1, SMAD2, SMAD5, SMAD8, TGFβ1, TGFβ2, TGFβ3 and/or BMP4 is then compared with a level of SMAD1, SMAD2, SMAD5, SMAD8, TGFβ1, TGFβ2, TGFβ3 and/or BMP4 that corresponds to a particular stage of ALS, in order to determine the stage of the disease in the subject. By way of example, elevated levels of BMP4 are detected in late stages of disease progression.

It is understood that differential expression patterns for SMAD1, SMAD2, SMAD5, and/or SMAD8 that correspond to different stages of disease can be used for diagnosis, prognosis, staging and/or treatment monitoring. Similarly, differential expression patterns for TGFβ1, TGFβ2, TGFβ3 and/or BMP4 that correspond to different stages of disease can be used for diagnosis, prognosis, staging and/or treatment monitoring. In another example, differential expression patterns for SMAD1, SMAD2, SMAD5, SMAD8, TGFβ1, TGFβ2, TGFβ3 and/or BMP4 that correspond to different stages of disease can be used for diagnosis, prognosis, staging and/or treatment monitoring. For example, an expression pattern showing increased levels of TGFβ1, TGFβ2 and TGFβ3, relative to levels BMP4, as compared to a control sample could be indicative of an earlier stage of ALS or disease onset. This could be useful for detecting presymptomatic stages of ALS prior to loss of motor function. In another example, an expression pattern showing increased levels of BMP4 relative to levels of TGFβ1, TGFβ2, TGFβ3, as compared to a control sample, could be indicative of a later stage of the disease.

SMAD expression patterns can be used in making a differential diagnosis, for example to differentiate between peripheral nerve injury and ALS. For example, progressive increases in SMAD1, SMAD5 and SMAD8 are observed over time for both peripheral nerve injury and ALS. However, whereas the levels of SMAD1, SMAD5 and SMAD8 remain elevated throughout progression of ALS, the levels of SMAD1, SMAD5 and SMAD8 revert to baseline levels upon reinnervation in peripheral nerve injury. Also, whereas SMAD8 increases to a greater extent than SMAD1 and SMAD5 in ALS, SMAD8 increases to a lesser extent than SMAD1 and

SMAD5 in peripheral nerve injury. Further, in ALS, a gradual rise in SMAD8 expression parallels disease progression, i.e., SMAD8 levels are higher at later stages of disease, whereas SMAD8 expression increases at the onset of peripheral nerve injury but reverts to baseline levels with reinnervation in peripheral nerve injury. In another example, increases in phosphorylated SMAD and increases in the phosphorylated SMAD (p-SMAD)/total SMAD (t-SMAD) ratio are observed over the course of ALS with the p-SMAD/t-SMAD ratio being significantly higher at the end-stage of ALS, as compared to earlier stages of ALS. In contrast, p-SMAD increases in the early stages of peripheral nerve injury and remains elevated, while the p-SMAD/t-SMAD ratio is only increased with reinnervation in peripheral nerve injury, indicating that SMAD activation persists in the muscle reinnervation phase. Thus, for any of the methods described herein, patterns of SMAD expression (e.g., levels of specific SMADs, time course of elevation of one or more SMADs, or ratios of phosphorylated to total SMAD for one or more SMADs, and time course of activation) can be determined instead of absolute levels of SMAD or absolute levels of nucleic acids encoding SMAD. Such patterns are useful in diagnosing, determining progression, assessing the success of treatment paradigms and the like.

Any of the methods provided herein for diagnosing or determining the progression of ALS can be used in combination with other methods for diagnosing ALS, including, but not limited to, neurologic examination, assessment of muscle strength, electromyography, nerve conduction studies and magnetic resonance imaging.

Further provided is a method of treating a subject with or at risk of developing ALS. The method comprises isolating a sample from the subject with ALS or at risk of developing ALS; detecting the level of SMAD1, SMAD2, SMAD5, and/or SMAD8 in the sample, an increase in SMAD1, SMAD2, SMAD5, and/or SMAD8, as compared to a control, indicating the subject has or is at risk for developing ALS; and administering an effective amount of an agent that treats ALS (e.g., riluzole (Rilutek)) or an agent that decreases the level of SMAD1, SMAD2, SMAD5, and/or SMAD8 in the subject.

Further provided is a method of treating a subject with or at risk of developing ALS. The method comprises isolating a sample from the subject with ALS or at risk of developing ALS and detecting the level of phosphorylation of SMAD1, SMAD2, SMAD5 and/or SMAD8. An increase in the level of phosphorylation as compared to a control, indicating the subject has or is at risk for developing ALS; and administering an effective amount of an agent that treats ALS (e.g., riluzole (Rilutek)) or an agent that decreases the level of phosphorylation of SMAD1, SMAD2, SMAD5, and/or SMAD8 in the subject.

Further provided is a method of treating a subject with or at risk of developing ALS. The method comprises isolating a sample from the subject with ALS or at risk of developing ALS and detecting the p-SMAD/t-SMAD ratio. An increase in the p-SMAD/t-SMAD ratio as compared to a control, indicating the subject has or is at risk for developing ALS; and administering an effective amount of an agent that treats ALS (e.g., riluzole (Rilutek®)) or an agent that decreases the p-SMAD/t-SMAD ratio in the subject.

Further provided is a method of treating a subject with or at risk of developing ALS. The method comprises isolating a sample from the subject with ALS or at risk of developing ALS; detecting the level of TGFβ1, TGFβ2, TGFβ3 and/or BMP4 in the sample, an increase in TGFβ1, TGFβ2, TGFβ3 and/or BMP4, as compared to a control, indicating the subject has or is at risk for developing ALS; and administering an effective amount of an agent that treats ALS (e.g., riluzole (Rilutek®)) or an agent that decreases the level of TGFβ1, TGFβ2, TGFβ3 and/or BMP4 in the subject.

Further provided is a method of treating a subject with or at risk of developing ALS. The method comprises isolating a sample from the subject with ALS or at risk of developing ALS; detecting the level of SMAD1, SMAD2, SMAD5, SMAD8, TGFβ1, TGFβ2, TGFβ3 and/or BMP4 in the sample, an increase in SMAD1, SMAD2, SMAD5, SMAD8, TGFβ1, TGFβ2, TGFβ3 and/or BMP4, as compared to a control, indicating the subject has or is at risk for developing ALS; and administering an effective amount of an agent that decreases the level of SMAD1, SMAD2, SMAD5, SMAD8, TGFβ1, TGFβ2, TGFβ3 and/or BMP4 in the subject.

Also provided is a method for determining the efficacy of a selected treatment for ALS in a subject. The method includes isolating a first sample from the subject before the selected treatment; detecting a first phosphorylation level of SMAD1, SMAD2, SMAD5, and/or SMAD8 in the sample; treating the subject with the selected treatment; isolating a second sample from the subject after the selected treatment; detecting the second phosphorylation level of SMAD1, SMAD2, SMAD5, and/or SMAD8 in the second sample; comparing the first and second levels of phosphorylated SMAD1, SMAD2. SMAD5, and/or SMAD8, a decrease in the level of phosphorylated SMAD1, SMAD2, SMAD5, and/or SMAD8 detected in the second sample indicating that the selected treatment is effective for treating ALS in the subject. The selected treatment is altered by one of skill in the art if no decrease in the second sample is detected. Such an alteration could include selecting a different therapeutic agent or change in dose or frequency of the same agent.

Also provided is a method for determining the efficacy of a selected treatment for ALS in a subject. The method includes isolating a first sample from the subject before the selected treatment; detecting a first p-SMAD/t-SMAD ratio in the sample; treating the subject with the selected treatment; isolating a second sample from the subject after the selected treatment; detecting the second p-SMAD/t-SMAD ratio in the second sample; comparing the first and second p-SMAD/t-SMAD ratio, a decrease in the p-SMAD/t-SMAD ratio detected in the second sample indicating that the selected treatment is effective for treating ALS in the subject. The selected treatment is altered by one of skill in the art if no decrease in the second sample is detected. Such an alteration could include selecting a different therapeutic agent or change in dose or frequency of the same agent.

Also provided is a method for determining the efficacy of a selected treatment for ALS in a subject. The method includes isolating a first sample from the subject before the selected treatment; detecting the a first level of SMAD1, SMAD2, SMAD5, and/or SMAD8 in the sample; treating the subject with the selected treatment; isolating a second sample from the subject after the selected treatment; detecting the second level of SMAD1, SMAD2, SMAD5, and/or SMAD8 in the second sample; comparing the first and second levels of SMAD1, SMAD2. SMAD5, and/or SMAD8, a decrease in the level of SMAD1, SMAD2, SMAD5, and/or SMAD8 detected in the second sample indicating that the selected treatment is effective for treating ALS in the subject. The selected treatment is altered by one of skill in the art if no decrease in the second sample is detected. Such an alteration could include selecting a different therapeutic agent or change in dose or frequency of the same agent.

Also provided is a method for determining the efficacy of a selected treatment for ALS in a subject that includes the steps of (a) isolating a first sample from the subject before the selected treatment; (b) detecting the level of TGFβ1, TGFβ2, TGFβ3 and/or BMP4 in the first sample; (c) treating the subject with the selected treatment; (d) isolating a second sample from the subject after the selected treatment; (e) detecting the second level of TGFβ1, TGFβ2, TGFβ3 and/or BMP4 in the second sample of step (d); (f) comparing the level of TGFβ1, TGFβ2, TGFβ3 and/or BMP4 detected in step (b) and (e), a decrease in the level of TGFβ1, TGFβ2, TGFβ3 and/or BMP4 detected in step (e) indicating that the selected treatment is effective for treating ALS in the subject. The selected treatment is altered by one of skill in the art if no decrease in the second sample is detected. Such an alteration could include selecting a different therapeutic agent or change in dose or frequency of the same agent.

Also provided is a method for determining the efficacy of a selected treatment for ALS in a subject that includes the steps of (a) isolating a first sample from the subject before the selected treatment; (b) detecting the first level of SMAD1, SMAD2, SMAD5, SMAD8, TGFβ1, TGFβ2, TGFβ3 and/or BMP4 in the first sample; (c) treating the subject with the selected treatment; (d) isolating a second sample from the subject after the selected treatment; (e) detecting a second level of SMAD1, SMAD2, SMAD5, SMAD8, TGFβ1, TGFβ2, TGFβ3 and/or BMP4in the sample of step (d); (f) comparing the first and second levels of SMAD1, SMAD2, SMAD5, SMAD8, TGFβ1, TGFβ2, TGFβ3 and/or BMP4 detected in step (b) and (e), a decrease in the level of SMAD1, SMAD2, SMAD5, SMAD8, TGFβ1, TGFβ2, TGFβ3 and/or BMP4 detected in step (e) indicating that the selected treatment is effective for treating ALS in the subject. The selected treatment is altered by one of skill in the art if no decrease in the second sample is detected. Such an alteration could include selecting a different therapeutic agent or change in dose or frequency of the same agent.

As used throughout, treat, treating, and treatment refer to a method of reducing or delaying one or more effects or symptoms of ALS. The subject can be diagnosed with ALS or suspected of having ALS. Treatment can also refer to a method of reducing the underlying pathology rather than just the symptoms. The effect of the administration to or treatment of the subject can have the effect of, but is not limited to, reducing one or more symptoms of the disease or disorder, a reduction in the severity of the disease or disorder, the complete ablation of the disease or disorder, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

In the methods provided herein, any agent that can be used for treating ALS can be administered, including, but not limited to agents that decrease the amount or activity of SMAD1, SMAD2, SMAD5, SMAD8 TGFβ1, TGFβ2, TGFβ3 and/or BMP4. For example, riluzole (Rilutek) can be administered to the subject. Agents that decrease the amount or activity of SMAD1, SMAD2, SMAD5, SMAD8 TGFβ1, TGFβ2, TGFβ3 and/or BMP4 include, but are not limited to, a chemical, small molecule, drug, protein, cDNA, antibody, shRNA, siRNA, miRNA, morpholino, antisense RNA, ribozyme or any other compound. Any of the therapeutic agents described herein can be administered in combination with, Baclofen to relieve stiffness in the subject, phenytoin to ease cramps, antidepressants or cell-derived neurotrophic growth factor, to name a few. Any of the treatment methods provided herein can also be combined with physical therapy and/or speech therapy.

The agents described herein can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Loyd V. Allen et al, editors, Pharmaceutical Press (2012)

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

In an example in which a nucleic acid is employed, the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). Physical transduction techniques can also be used, such as liposome delivery and receptor-mediated and other endocytosis mechanisms (see, for example, Schwartzenberger et al., Blood 87:472-478, 1996) to name a few examples. These methods can be used in conjunction with any of these or other commonly used gene transfer methods.

Administration can be carried out using therapeutically effective amounts of the agents described herein for periods of time effective to treat ALS. The effective amount can be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent can be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration can be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment, for example by topical application or local injection. Multiple administrations and/or dosages can also be used. Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLE I

### Animals

B6.Cg-Tg (SOD1*G93A) 1 Gur/J mice were purchased from The Jackson Laboratory (Bar Harbor, Me.). Transgenic mice were maintained in the hemizygous state by mating G93A males with C57BL/6J females. Non-transgenic littermates were used as controls. For the sciatic nerve injury experiment, mice were from the C57BL/6J background and bred in-house. All animal procedures were reviewed and approved by the UAB Institutional Animal Care and Use Committee in compliance with the National Research Council Guide for the Care and Use of Laboratory Animals. For sciatic nerve injury, the spared nerve injury (SNI) model was chosen whereby all divisions of the sciatic nerve at the trifurcation, except the sural branch, are transected (Shields et al., “Spared nerve injury model of neuropathic pain in the mouse: a behavioral and anatomic analysis,” J Pain. 2003;4:465-70). Under oxygen/isoflurane anesthesia, mice in this experiment received unilateral SNI on the left side. A control group underwent sham surgery in which the sciatic nerve was exposed but not transected.

### Behavioral Assessment

For the G93A SOD1 mice, clinical progression was evaluated by weight determination and performance on the rotarod (San Diego Instruments, San Diego, Calif.). End stage disease was determined when the mouse could right itself after 30 seconds when placed on its side. Rotarod testing was based on previously published methods (Kaspar et al. “Retrograde viral delivery of IGF-1 prolongs survival in a mouse ALS model,” Science 2003;301:839-42). Briefly, the rod was rotated at a gradually accelerating speed up to 11 rpm over a 2 min interval, and the animal's latency to fall was registered automatically. Measurements were collected once a week in a cohort of 20 transgenic SOD1*G93A mice and 20 wild-type littermate controls. For animals undergoing SNI, pain threshold testing with the Von Frey test was used to confirm nerve injury (“Chaplan et al. “Quantitative assessment of tactile allodynia in the rat paw,” J Neurosci Methods 1994;53:55-63). Briefly, the up-down method was used to estimate the 50% withdrawal thresholds using nylon monofilaments (Stoelting Co., Wood Dale, Ill.). Mice were placed in custom-made Plexiglas cubicles (5×8.5×6 cm) on a perforated metal sheet and were permitted to habituate for at least 1 hour prior to testing. As the sural nerve was spared, the filaments were applied to the lateral aspect of the hindpaw for 1 second and responses were recorded. Two consecutive measures were taken on both hindpaws at each time point, but only the measures on the ipsilateral paw are shown. All mice (n=6) were tested at baseline and one week post nerve injury (PNI). To determine the extent of motor deficits, mice were assessed 2 days PNI using the Basso Mouse Scale for Locomotion (BMS) (Basso et al. “Mouse Scale for locomotion detects differences in recovery after spinal cord injury in five common mouse strains,” J Neurotrauma 2006;23:635-59). This ten point scale was assessed by two independent observers blinded to the experimental condition. A score of 0 indicates no ankle movement and 10 is normal locomotion.

### Tissue Collection

After approval by the UAB Institutional Review Board, the UAB Nerve and Muscle Histopathology Laboratory database was searched for archived biopsy samples (stored at −80° C.) representative of ALS, normal and diseased controls (myopathy and neuropathy). A cohort of 27 patients with ALS was identified (Table 1). At the time of biopsy, 17 patients had clinically probable or definite ALS by revised El Escorial criteria (Brooks et al. “El Escorial revisited: revised criteria for the diagnosis of amyotrophic lateral sclerosis,” Amyotroph Lateral Scler. 2000;1:293-9. The remaining 10 patients had possible ALS, but developed a definite diagnosis over time.

For mouse samples, animals were sacrificed by CO₂ inhalation followed by cervical dislocation. Brain, spinal cord, hindlimb and forelimb muscle tissues were dissected at discrete time points between 40-150 days post-natal.

Samples were briefly rinsed in phosphate-buffered saline (PBS), and frozen in liquid nitrogen and stored at −80 C prior to biochemical analysis. For immunohistochemical studies, samples were frozen on dry ice in Tissue-Tek compound or fixed for one day in 4% paraformaldehyde in 0.1 M sodium phosphate buffer, pH 7.4.

### Western Blot, and Immunohistochemistry

Tissues were homogenized in T-Per (Pierce Endogen, Rockford, IL) and quantitated with a bicinchoninic acid (BCA) protein assay kit. Sixty micrograms of protein were subjected to SDS-polyacrylamide gel electrophoresis, blotted and probed with antibodies to the following targets: p-Smad 1/5/8 (Cell Signaling, Beverly, Mass.), t-Smad 1/5/8 (Santa Cruz Biotechnology, Paso Robles, Calif.), and GAPDH (Cell Signaling). Densitometry was done with the VersaDoc Imaging System (Bio-Rad, Hercules, Calif.) and quantified using Image Lab (Bio-Rad). For immunohistochemistry, 10 micron sections of muscle were fixed in Bouin's fixative for 15 min and then oxidized in 0.3% H₂O₂ for 15 min. After blocking, sections were incubated with p-Smad or t-Smad 1/5/8 antibodies (1:10) overnight at 4° C. Slides were incubated for an hour at RT with donkey anti-rabbit secondary antibody conjugated with horseradish peroxidase-labeled polymer (Jackson ImmunoResearch, West Grove, Pa.). Slides were incubated in TSA Plus Cyanine 3 (PerkinElmer, Waltham, Mass.) at 1:1,500 for 30 min, Wheat Germ Agglutinin (WGA), Alexa Fluor® 488 (Invitrogen, Carlsbad, Calif.) at 1:200 for 10 min, followed by Hoechst 33342 (Sigma-Aldrich, St. Louis, Mo.) at 1:20,000 for 10 min. The prepared slides were viewed under an Olympus DP 71 fluorescence microscope.

RNA Isolation, Next-Generation Sequencing, and qRT-PCR

RNA was extracted from frozen tissues with Trizol Reagent (Invitrogen, Waltham, Mass.) according to the manufacturer's instructions. Next-Generation sequencing was performed on ALS and disease control muscle RNA samples by the UAB Genomics Core Facility. Transcriptional sequencing methods were performed essentially as described in Mortazavi et al. “Mapping and quantifying mammalian transcriptomes by RNA-seq.,” Nat Methods 2008; 5:621-8; and Bentley et al. “Accurate whole human genome sequencing using reversible terminator chemistry,” Nature 2008;456:53-9. For qRT-PCR, two micrograms of total RNA were reverse transcribed according to the manufacturer's specifications (Applied Biosystems, Carlsbad, Calif.). Multiplex PCR was done using On Demand Taqman primers (Applied Biosystems) in a ViiA 7 Real Time PCR System (Applied Biosystems). Probes for all target genes were FAM labeled, and the endogenous control GAPDH was labeled with VIC dye/MGB. A standard thermal cycle protocol was used. Data were analyzed with the ViiA 7 Software version v1.1. The baseline was auto set, and threshold values were adjusted from 0.09 to 0.2 based on the amplification curve of different targets. Quantification of target mRNAs was done by the AACT method using GAPDH as an internal control (Livak et al. “Analysis of relative gene expression data using real-time quantitative PCR and the 2(-Delta Delta C(T)) Method,” Methods 2001;25:402-8).

### Statistical Analyses

For qRT-PCR comparisons between ALS group and all other groups, one-way ANOVA and pair-wise t tests were conducted on ACt values. For Western blot data analysis, the data was logarithm-transformed and normalized using the GAPDH values for each lane. Protein quantities were modeled using a linear model with genotype, age and their interaction term controlling for blot. For the p-Smad/t-Smad analysis, ratios on the logarithm scale were modeled using a linear model similar to that for the total amount of protein. Paired t tests were used to compare baseline and post nerve injury mechanical sensitivity, and unpaired t tests were used to compare protein or RNA across groups.

### Results

Smad 1,5 and 8 mRNAs are Elevated in Muscle Biopsies of Patients with ALS

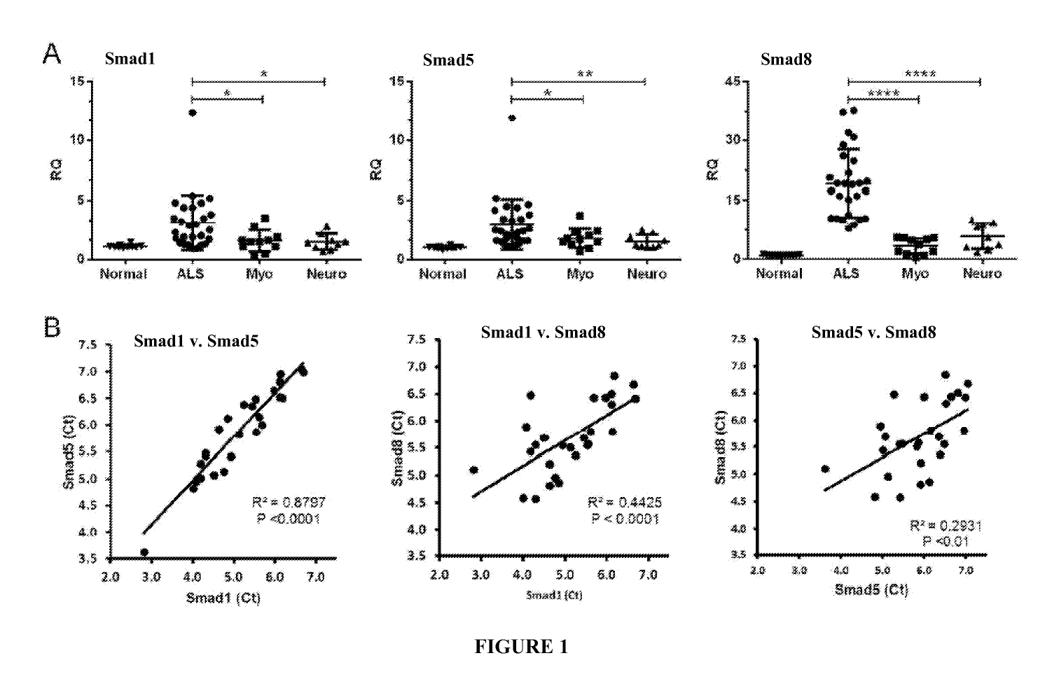

Next-generation sequencing of RNA was performed on RNA isolated from muscle biopsies of patients with ALS, diseased controls and normal controls. A significant (4-8-fold) elevation of Smad8 mRNA was observed in ALS muscle samples over controls. To validate this finding, a large cohort of ALS and control muscle samples were tested by qRT-PCR (Tables 1 and 2). The ALS cohort (n=27) reflected demographics that matched the overall ALS population, including a mean age of 61 years at diagnosis, and a slight male predominance. Medical Research Council (MRC) grades were available for all muscles and ranged from 0 to 5. EMG testing of the biopsied muscle was available for 25 patients and all showed evidence of active and/or chronic denervation. The control population for these studies consisted of histologically normal patients (referred for non-specific complaints, usually myalgias or cramps), and diseased controls (histologically proven myopathy or neuropathy-related denervation). Smad8 mRNA levels were assessed by qRT-PCR in these samples, using GAPDH as an internal control (FIG. 1A). A significant upregulation of Smad8 was found in ALS patients at 19 fold compared to normals, 3-fold to neuropathy and 5.6-fold to myopathy controls (P<0.0001 for all comparisons). Upon looking at closely related family members, significant increases in Smad 1 and 5 were found, but to a much smaller degree (˜3-fold over controls and ˜1.5-fold over diseased controls, P<0.05). Correlation between all Smads in the ALS samples was highly significant (FIG. 1B), with Smad1 and 5 being the strongest (Pearson coefficient of 0.88, P<0.0001). Smad mRNA levels and MRC grade showed a positive correlation trend which was not statistically significant at the 0.05 level (p values 0.09 to 0.23).

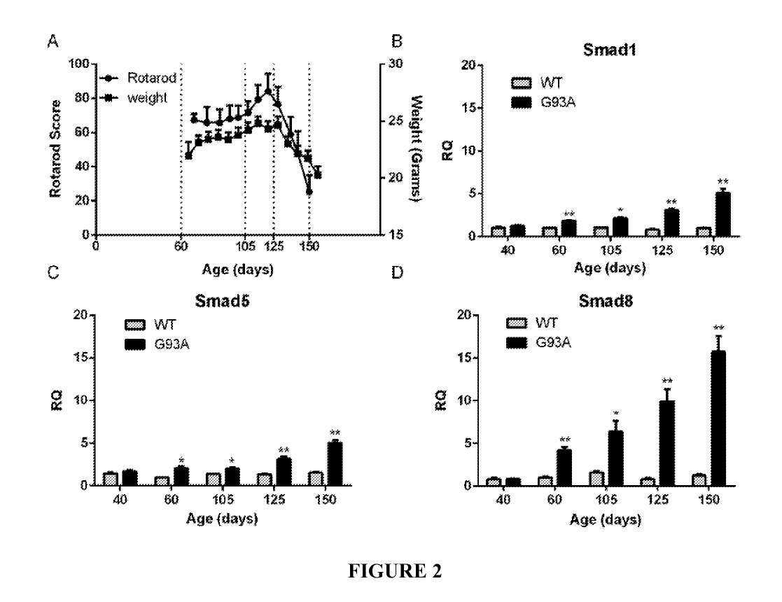

Smad 1,5 and 8 mRNAs are elevated in muscle from G93A SOD1 mice As a separate validation, expression of Smad mRNA in muscle samples from the G93A SOD1 mouse was examined. This model recapitulates many of the pathological and clinical features of ALS, including progressive weakness, motor neuron loss, and muscle denervation (Gurney et al. “Motor neuron degeneration in mice that express a human Cu,Zn superoxide dismutase mutation,” Science 1994;264:1772-5). The mouse model for these studies is on the C57/BL6 background and displays a later onset of clinical symptoms and longer survival than the original BL6/SJL mouse (FIG. 2A) (Heiman-Patterson et al. “Background and gender effects on survival in the TgN(SOD1-G93A)1Gur mouse model of ALS,” J. Neurol Sci. 2005;236:1-7). In this model, subtle differences in motor function between controls can be detected 45 days (post natal), well before overt clinical manifestations which usually occur at 90-100 days (Hayworth et al. “Pre-symptomatic detection of chronic motor deficits and genotype prediction in congenic B6.SOD1(G93A) ALS mouse model,” Neuroscience 2009;164:975-85.) By weeks 7-8, there is a significant decrease in the weight of hindlimb muscles accompanied by increased expression of proteins associated with stress response, oxidative metabolism, and autophagy. The gastrocnemius muscle was sampled at different clinical stages, including three pre-clinical stages (40, 60 and 105 days), one just after onset of motor deterioration as measured by rotarod testing (125 days) and one at end-stage (150 days). Wild-type (WT) littermates served as a control. RNA was extracted and assessed by qRT-PCR.

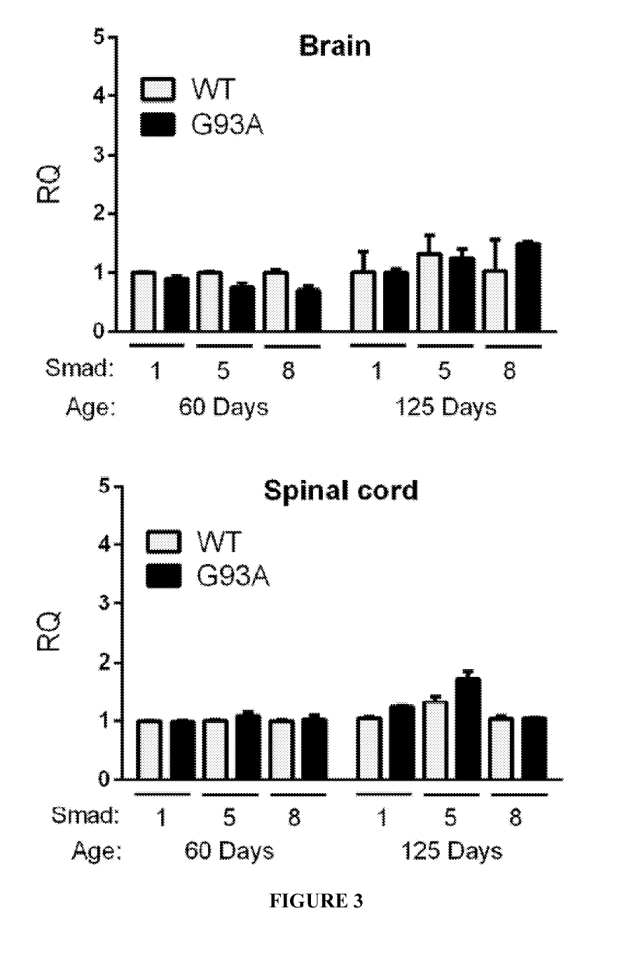

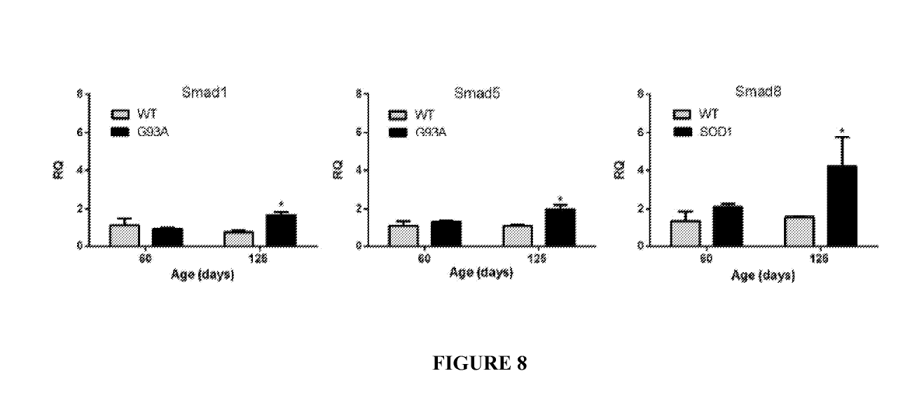

A pattern of upregulation very similar to the human samples was observed with Smad8 showing a substantially greater fold-change over WT mice (up to 17-fold at end-stage) compared to Smad1 and 5 (up to 5-fold at end-stage). There was a clinical stage-dependent increase in all Smads, beginning in pre-symptomatic stages of the disease (60 and 105 days). At day 40, however, there was no difference between ALS and control samples. These markers were assessed in forelimb muscles which have a slower onset of denervation, atrophy and functional impairment. No elevation in any of the Smad mRNAs was observed at day 60 but a significant increase was observed at day 125 (FIG. 8). The increase was less than the hindlimb muscles by 2-4 fold. To determine whether Smad upregulation was specific to muscle or a more global phenomenon, mouse brain and spinal cord tissues were assessed at pre-symptomatic (60 days) and symptomatic (125 days) stages. No differences were observed between WT and mutant mice (FIG. 3). These findings indicate that Smad1, 5 and 8 upregulation is an early and specific event in the muscle of ALS mice, and that the gradual rise in gene expression, particularly Smad8, parallels disease progression.

### Smad 1,5, 8 Protein is Elevated in Muscle From G93A SOD1 Mice and Human ALS Patients

Since the data indicated a significant upregulation of Smad1, 5, and 8 mRNA levels, it was next determined whether Smad protein was increased and/or activated.

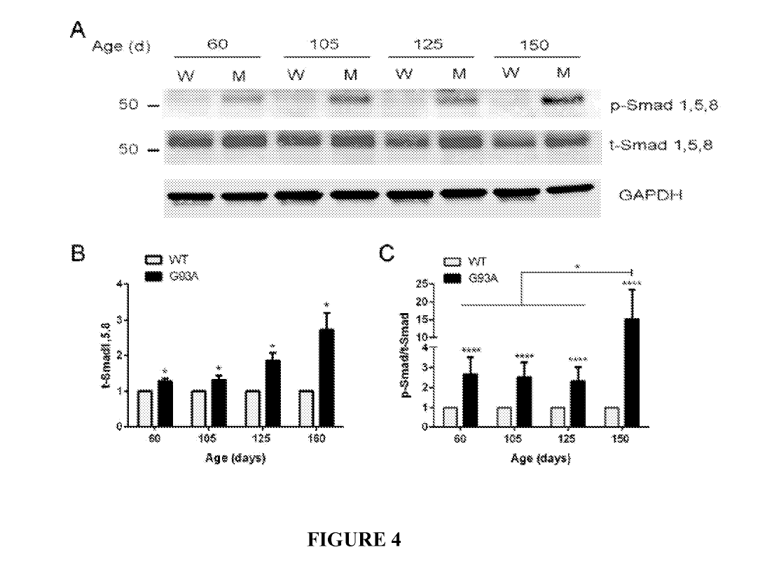

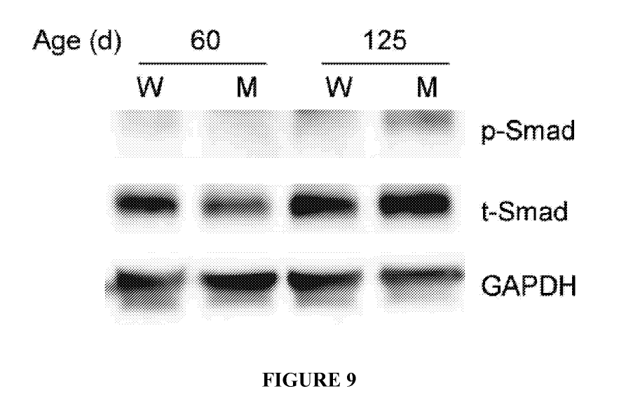

Western blot analysis of mouse muscle samples was performed using an antibody which recognizes the phosphorylated (activated) form of all three proteins. An increase in activated Smad (p-Smad) was observed at each of the stages, most prominently at 150 days (representative blot shown in FIG. 4A). Re-probing the blot with an antibody that recognizes total Smad1,5,8 (t-Smad), however, showed an increase in total levels consistent with the mRNA expression patterns. To quantify changes both in total Smad levels and p-Smad/t-Smad ratios, three to six independent mutant and WT littermates per clinical stage were assessed. A significant increase in total Smad1,5,8 protein over WT control was observed over the course of the disease (P<0.05; FIG. 4B). The fold-difference between mutant and WT mice progressively increased, and at end-stage it was nearly 3-fold. Despite the increase in total Smad protein, however, the p-Smad/t-Smad ratio was still higher than WT at each clinical stage (FIG. 4C). At end-stage, the p-smad/t-smad ratio was nearly 15-fold greater than WT (P<0.0001) and significantly higher than earlier stages of the ALS mice (P<0.05). Forelimb muscles were also assessed for p-Smad and no differences were observed at day 60, but a clear increase was observed at day 125, consistent with a delayed onset in forelimb muscles (FIG. 9). In summary, Smad1,5,8 protein increased with disease progression in parallel with Smad activation.

p-Smad 1,5,8 Protein Localizes to Nuclei and Cell Membrane in Muscle from G93A SOD1 mice and human ALS

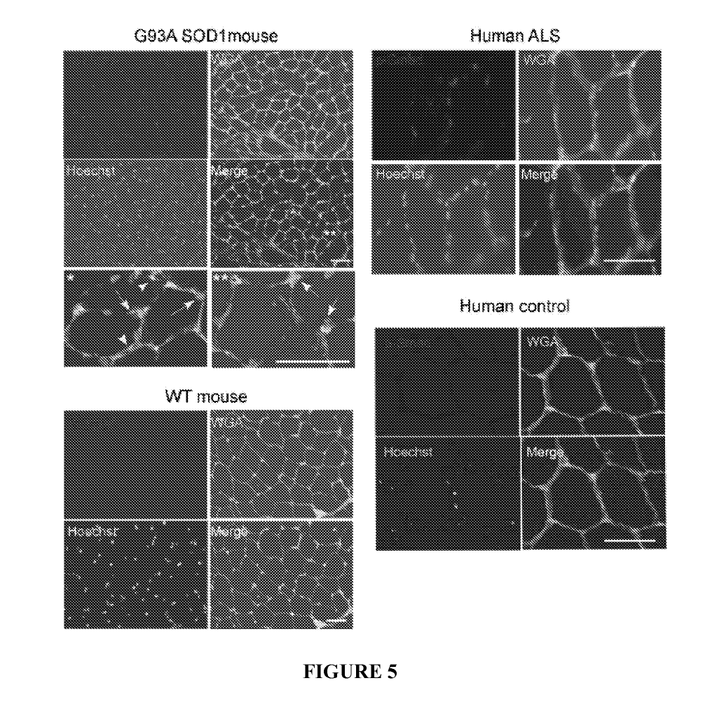

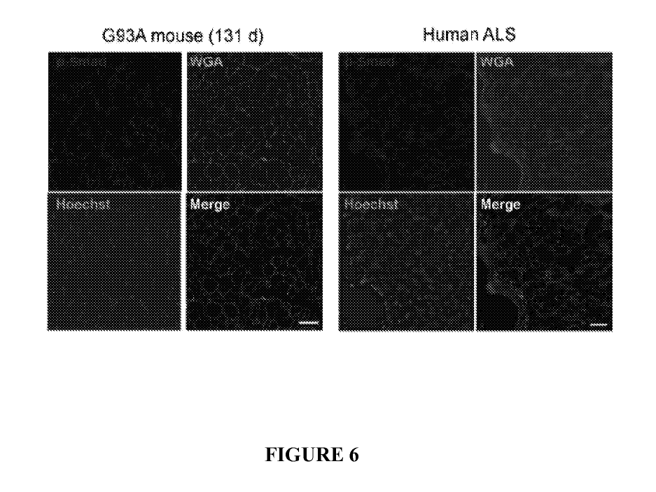

Localization of p-Smad1,5,8 by immunofluorescence in mouse at a preclinical stage (60 days) was assessed and staining was detected in multiple myofibers, but no staining was detected in an age-matched WT mouse (FIG. 5). There was both punctuate and more diffuse staining observed in the periphery of the cells. Higher power views showed overlap of the punctuate signal with Hoechst-stained myonuclei, indicating nuclear localization. Additionally, immunoreactivity was seen along the periphery of the myocytes which overlapped with WGA, a lectin stain which identifies myofiber membrane boundaries. Although the data suggest localization to the cell membrane, it cannot be excluded that this may be related to muscle atrophy and membrane leakiness. In a biopsy sample from a patient with ALS at an early stage, there was also specific localization of p-Smad to the myonuclei (FIG. 5). A muscle sample from an older mutant mouse (131 days) was also assessed and a similar pattern but with more extensive staining (FIG. 6) was found. This finding is consistent with Western blot results shown in FIG. 4. A muscle sample from an ALS patient at end stage also showed extensive p-Smad staining of myonuclei and myofiber borders suggesting a similar pattern of Smad1,5,8 accumulation with disease progression.

### Smads are Elevated in a Peripheral Nerve Injury Model

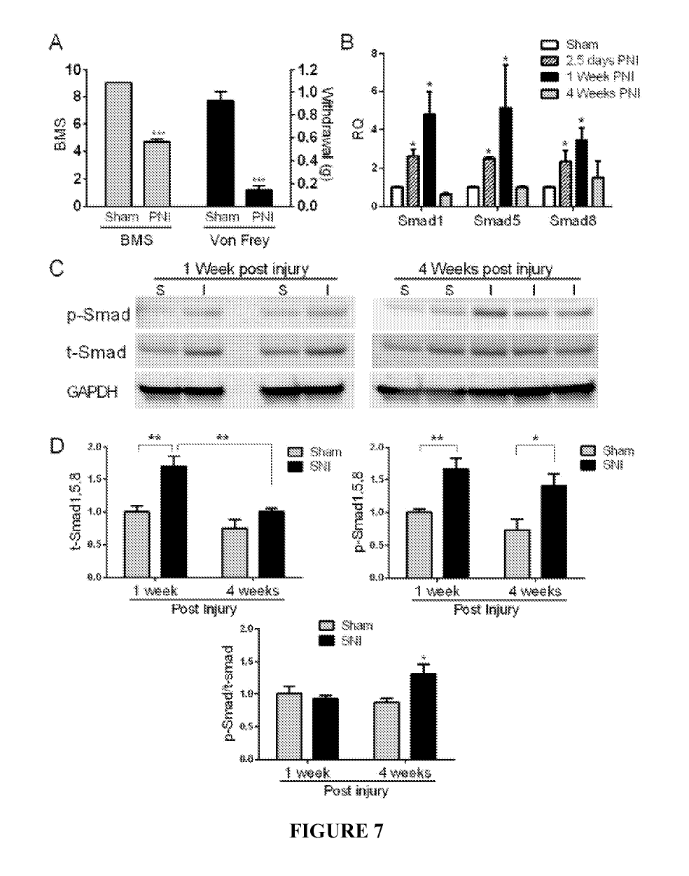

To assess Smad induction in non-ALS muscle denervation, a model of sciatic nerve injury was used. Mice underwent sciatic nerve transection or sham surgery and Smad profiles were assessed at 2.5 d, 1 and 4 weeks post nerve injury (PNI). Nerve injury was confirmed by motor defects using the Basso Mouse Scale for Locomotion (BMS) and the development of allodynia using the Von Frey withdrawal test (FIG. 7A). Injured mice showed significant locomotion defects (day 2 PNI) and mechanical allodynia (day 7 PNI). Smad mRNA expression was assessed in the gastrocnemius muscle in the injured limb. All comparisons were made to a control group which underwent a sham procedure where the sciatic nerve was exposed but not transected.

A progressive increase in Smad1 and 5 mRNAs to approximately 5-fold over control was observed at 1 week PNI which then returned to baseline at 4 weeks, a time point in which a majority of muscle fibers are reinnervated (FIG. 7B). Smad8 increased to a lesser extent (˜4-fold) and also reverted back toward baseline at 4 weeks. Assessment of protein expression by Western blot revealed an increase in total Smad1,5,8 at one week post injury (P<0.05) which then declined at 4 weeks to that of control (FIG. 7C and 7D). Phosphorylated Smad1,5,8 was significantly elevated at 1 week and remained elevated at 4 weeks, whereas the p-smad/t-smad ratio was only increased at 4 weeks. In summary, there was increased expression of the Smads to a similar degree following nerve injury, which reversed over time. Smad activation persisted in the muscle reinnervation phase out of proportion to total Smad expression.

As disclosed herein, Smad1, 5 and 8 were identified as muscle biomarkers of disease progression in ALS at the transcriptional (mRNA), translational (protein) and post-translational (phosphorylation) levels. Smad8, first identified by RNA sequencing of human ALS muscle biopsy samples, showed the highest specificity for ALS patients at the mRNA level (FIG. 1). The markers were validated in a SOD1 mouse model of ALS where there was a gradual accumulation of Smad protein and mRNA, starting in pre-clinical stages to end-stage. Concomitantly, there was activation of these Smad members which enhanced significantly as the disease progressed.

A challenge facing investigations of novel therapeutic compounds in ALS is the ability to monitor clinical responses in a timely manner to determine efficacy. Standard clinical testing, including the ALS Functional Rating Score, is relatively insensitive. Based on these findings, two features of Smads correlated with disease progression in the ALS mouse: gene upregulation (mRNA and protein) and protein activation (phosphorylation). These molecular changes also occurred before overt clinical manifestations, suggesting that the markers are sensitive to more subtle loss of motor neurons. Furthermore, the hindlimb muscles showed earlier expression and activation of Smads compared to forelimb muscles providing further support that these markers can track disease progression.

As shown herein, Smad8, Smad1 and Smad5 mRNAs were significantly elevated in human ALS muscle samples. The markers displayed a remarkably similar pattern in the G93A SOD1 mouse model of ALS with increases detected at pre-clinical stages. Expression at the RNA and protein levels as well as protein activation (phosphorylation) significantly increased with disease progression in the mouse. The markers were also elevated to a lesser degree in gastrocnemius muscle following sciatic nerve injury, but then reverted to baseline during the muscle reinnervation phase. These data indicate that Smad1,5,8 mRNA and protein levels, as well as Smad phosphorylation, are elevated in ALS muscle and could serve as markers of disease progression or regression. Further, these markers have utility in detecting arrest or reversal of muscle denervation prior to changes in motor function, which are advantageous when assessing efficacy of experimental therapies.

### EXAMPLE II

### Animals

B6.Cg-Tg (SOD1*G93A) 1 Gur/J mice were purchased from The Jackson Laboratory (Bar Harbor, Me.). Transgenic mice were maintained in the hemizygous state by mating G93A males with C57BL/6J females. Non-transgenic littermates were used as controls. For the G93A SOD1 mice, clinical progression was evaluated by weight determination and performance on the rotarod as described previously (Si et al., “Smads as muscle biomarkers in amyotrophic lateral sclerosis” Ann Clin Trans' Neurol. 1(10):778-87 (2014)). End stage disease was determined when the mouse could not right itself after 30 seconds when placed on its side. At this point animals were euthanized by CO₂ inhalation followed by cervical dislocation. All animal procedures were reviewed and approved by the UAB Institutional Animal Care and Use Committee in compliance with the National Research Council Guide for the Care and Use of Laboratory Animals.

### Tissue Collection and Cell Culture

The study was approved by the UAB Institutional Review Board. Muscle biopsy samples were identified in a database of archived samples from the neuromuscular division at UAB. The clinical details of patients with ALS, neuropathy, myopathy or no neuromuscular disease are described in our previous publication (Si et al.). C2C12 cells were grown in high glucose DMEM containing 10% FBS, 1% 100, and 1 mM sodium pyruvate. After splitting cells, the medium was changed to high glucose DMEM containing 2% horse serum, and 1 mM sodium pyruvate. Cells were treated with TGF-β1, 2 and 3 (R&D System, Minneapolis, Minn.) at 10 ng/ml.

### Western Blot, ELISA and Immunohistochemistry

For Western blot, tissues were homogenized in T-Per (Pierce Endogen, Rockford, Ill.) and quantitated with a bicinchoninic acid (BCA) protein assay kit. Sixty micrograms of protein were electrophoresed in an SDS-polyacrylamide gel, blotted and probed with antibodies to the following targets: TGF-β1 (Promega, Madison, Wis.), TGF-β2 (Santa Cruz Biotechnology, Paso Robles, Calif.), TGF-I33 (Abcam, Cambridge, Mass.), and GAPDH (Cell Signaling, Danvers, Mass.). Densitometry was done with the VersaDoc Imaging System (Bio-Rad, Hercules, Calif.) and quantified using Image Lab (Bio-Rad). For immunohistochemistry, ten micron paraffin sections and OCT sections were used. OCT slides were fixed in Bouin's fixative for 15 min. Deparaffinized sections were immersed in 10 mM citrate buffer (pH 6.0) heated at 100° C. for 30 min, and allowed to cool to room temperature. After fixation, all sections were treated with 3% H₂O₂ for 10 min. After blocking, sections were incubated with TGF-β1, 2 and 3 antibodies (1:50) overnight at 4° C. Slides were incubated for an hour at RT with donkey anti-rabbit secondary antibody conjugated with horseradish peroxidase-labeled polymer (Jackson ImmunoResearch, West Grove, Pa.). Slides were incubated in TSA Plus Cyanine 3 (PerkinElmer, Waltham, Mass.) at 1:1,500 for 30 min, Wheat Germ Agglutinin (WGA), Alexa Fluor® 488 (Invitrogen, Carlsbad, Calif.) at 1:400 for 20 min, and Hoechst 33342 (Sigma-Aldrich, St. Louis, Mo.) at 1:20,000 for 5 min. The prepared slides were viewed under a TCS SP5 Visible-Upright Confocal Microscope (Leica Microsystems, Buffalo Grove, Ill.). TGF-β1 quantities in mouse and human muscle lysates were determined using the human TGF-β1 Quantikine ELISA Kit (R&D System) according to the manufacturer's instructions. Samples were acid activated using a protocol provided by the manufacturer. Quantities were estimated based on a standard curve generated with recombinant TGF-β1.

RNA Isolation and qRT-PCR

RNA was extracted from frozen tissues with Trizol Reagent (Invitrogen, Waltham, MA) according to the manufacturer's instructions. Two micrograms of RNA were reverse transcribed according to the manufacturer's specifications (Applied Biosystems, Waltham, Mass.). Multiplex PCR was done using as previously described (Si et al.)

### Statistical Analyses

Comparisons between ALS group and controls were conducted using a Mann Whitney test. A Student's t test was used for analysis of mouse qRT-PCR and ELISA results. For Western blot analysis, we first normalized the densitometry values with the housekeeping control (GAPDH) for each lane. A paired t test was used for assessing densitometry comparisons within each blot, comparing wild-type to G93A samples at each age. Pearson correlation coefficients were calculated using Graphpad software (San Diego, Calif.).

### Results

### TGF-β1, 2 and 3 mRNAs are elevated in muscle biopsies of patients with ALS

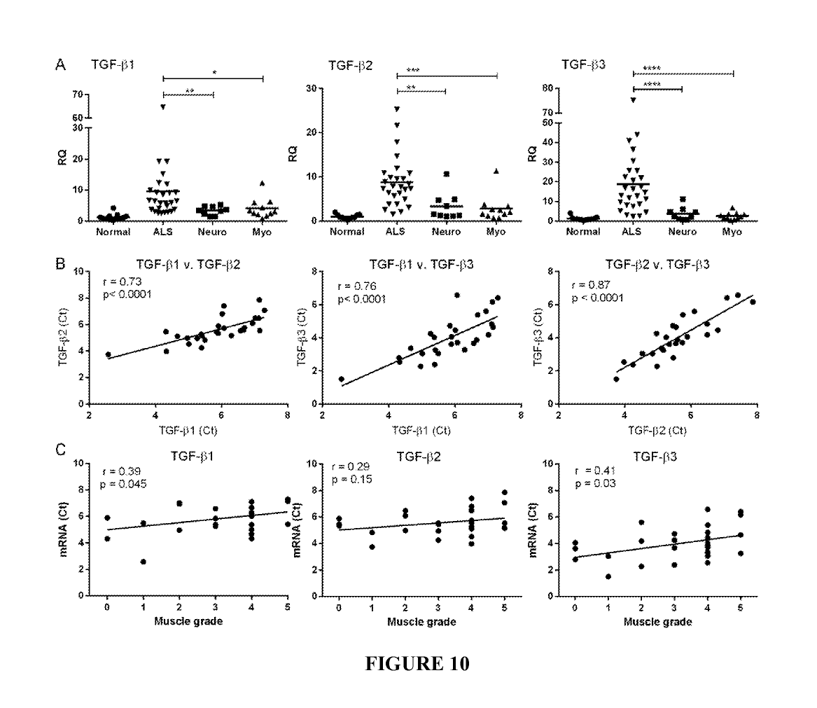

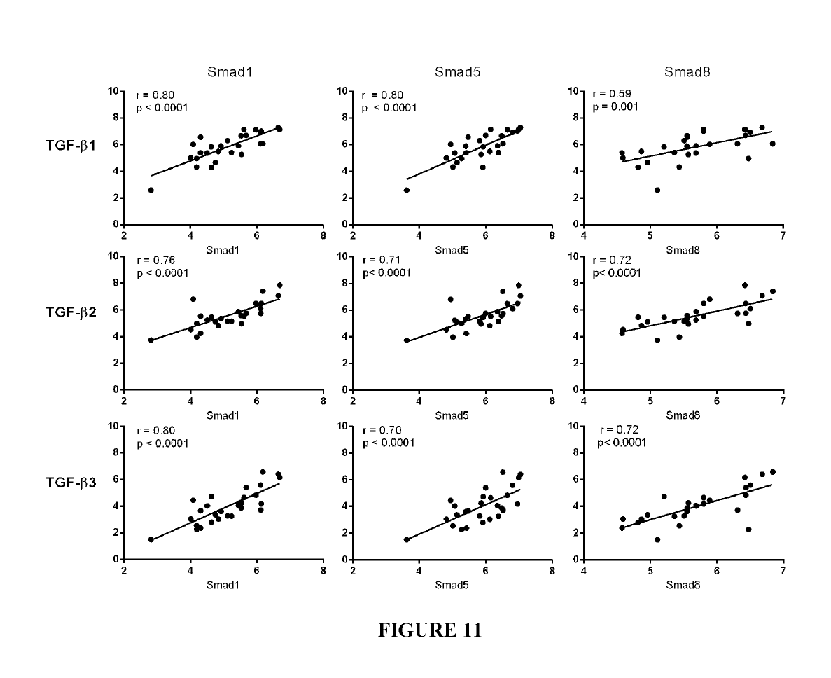

As set forth above, phosphorylation (activation) and upregulation of Smads1, 5 and 8 was observed in ALS muscle tissue. These signal transduction proteins are typically activated after engagement of a TGF-βligand to its cognate cell surface receptor. An analysis of RNA sequencing data (methodology as set forth above and in Si et al.) revealed a significant increase in TGF-β3 mRNA in ALS muscle biopsy samples. This target was assessed in a cohort of 27 ALS patients and expression levels were compared to controls (FIG. 10A). Clinical descriptions of the cohort and controls are described in Si et al. Briefly, the ALS group had a mean age of 61 years, equally divided between males and females, with 25% bulbar and 75% spinal onset. Myopathy controls consisted of patients with polymyositis and mitochondrial myopathy; neuropathy controls included patients with axonal and demyelinating peripheral neuropathies. A 15-fold increase over normal controls and a 5 to 7-fold increase over neuropathy and myopathy controls (p<0.0001) was observed. TGF-β1 and 2 mRNAs, although not identified by RNA sequencing, also increased (2 to 3-fold) over diseased controls. There was significant correlation among the different isoforms in the ALS samples (p<0.0001; FIG. 10B). An inverse correlation was observed between muscle grade (Medical Research Council scale) of the biopsied muscle and TGF-β1 and 3 mRNA levels (FIG. 10C). Next, Smad mRNA levels were compared to TGF-βand striking positive correlations between Smads 1, 5 and 8 were found with each isoform (FIG. 11). Pearson coefficient values were 0.70 or higher for all but one of the comparisons (p<0.0001). In summary, the RNA data show that TGF-β1, 2 and 3 mRNAs are significantly increased in muscle of ALS patients, and this expression pattern parallels that of the Smads.

### TGF-βmRNA and Protein are Increased at an Early Age in the G93A SOD1 Mouse

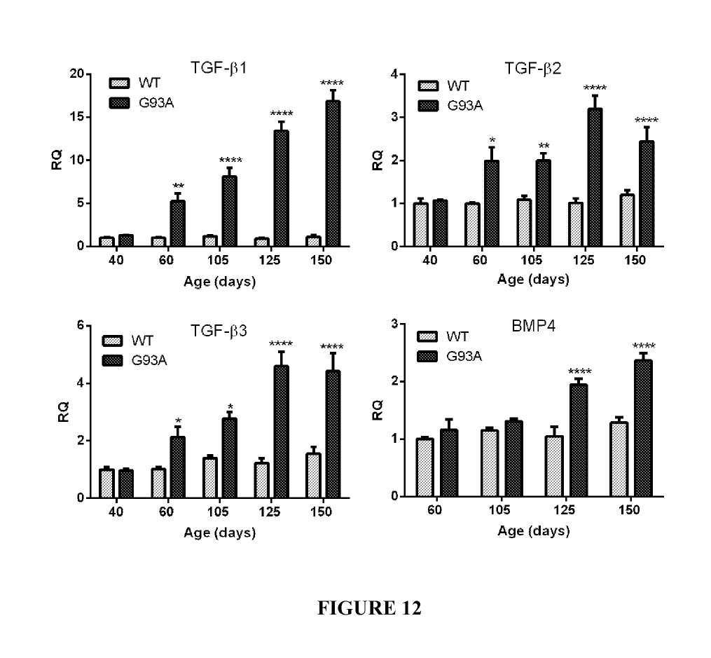

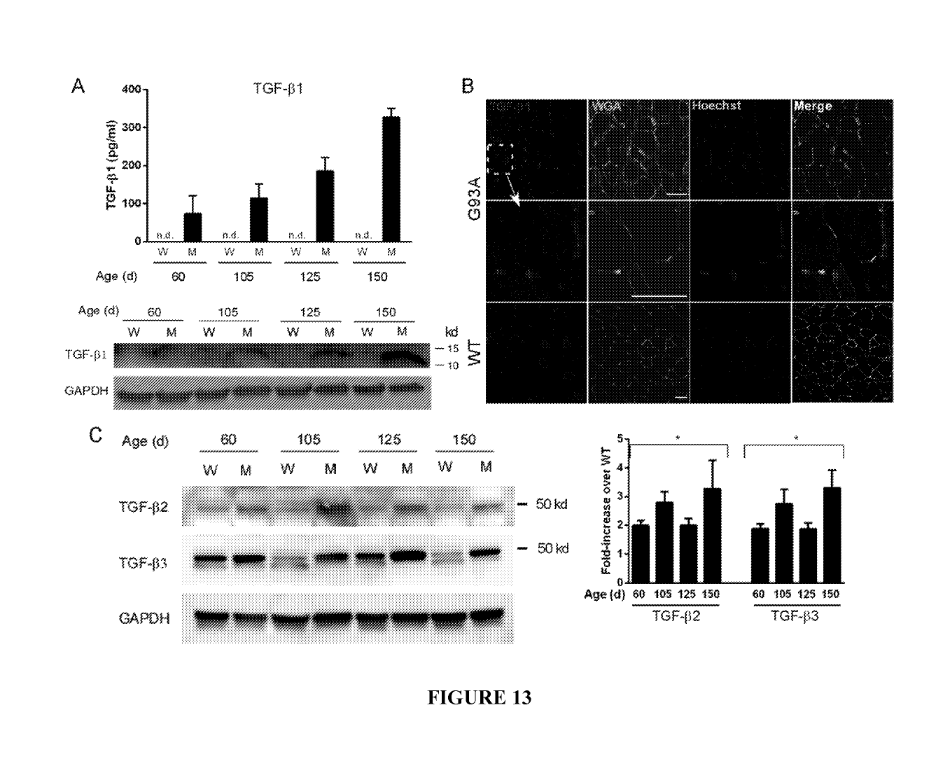

Whether TGF-βmRNA upregulation occurred in skeletal muscle from the G93A SOD1 mouse and if the temporal pattern of expression paralleled that of the Smads was determined. Induction of Smad1, 5 and 8 mRNAs was observed between post-natal day 40 and 60. At that time interval, no overt clinical manifestations are observed, but subtle motor deficits have been described (Hayworth et al. “Pre-symptomatic detection of chronic motor deficits and genotype prediction in congenic B6.SOD1(G93A) ALS mouse model. In the colony used for these studies, the animals have a longer survival time (mean of 161 days) and no gender effect. These features are similar to what has previously been reported for this model (Hayworth et al.) Further details regarding progression and disease onset are provided in Si et al. For all three TGF-βisoforms a significant increase in mRNA compared to littermate controls starting at 60 d and throughout the clinical course (FIG. 12) was observed. TGF-β1 showed the greatest increase at each time point (more than 15-fold by end-stage). There was no increase at 40 d in any of the isoforms indicating a temporal pattern similar to the Smads. BMP4, another TGF-βreceptor ligand which showed a non-significant upward trend in RNA sequencing analysis studies, increased only in the later stages (125 and 150 d) indicating that TGF-β1, 2 and 3 mRNA induction can be selective in the early stages of disease. Protein expression was also assessed. For TGF-β1, gradual increases were observed by ELISA with disease progression (FIG. 13A). Detection required acid activation indicating that the ligand is predominantly in the latent form. Western blot (under reducing conditions) showed a similar increase in the mature form of TGF-β1 with disease progression. Immunohistochemistry with a TGF-β1 antibody showed labeling of mononuclear cells adjacent to muscle borders as outlined by WGA staining (FIG. 13B). Little to no staining was observed in WT muscle. For TGF-β2 and 3, Western blot analysis showed an increase of pre-processed protein in mutant mice over control at each age toward end-stage. Densitometry of three independent mouse samples indicated a 2-3-fold increase over age-matched controls, with an upward trend at end-stage (FIG. 13C). Processed forms were not detected, and immunohistochemistry did not show consistent staining for either isoform.

### TGF-β1 Protein is Expressed in Human ALS Muscle

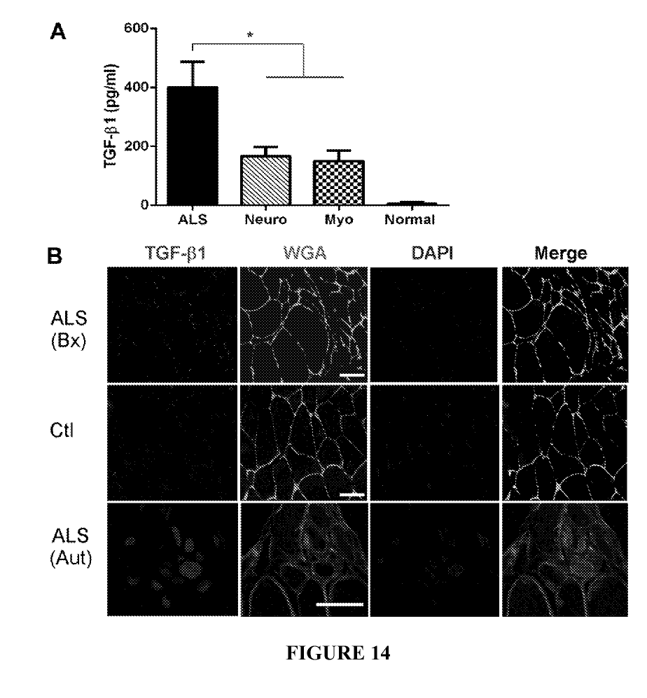

ELISA analysis of muscle lysates showed a marked increase in TGF-β1 in human ALS samples that was greater than 2-fold over disease controls (FIG. 14A). Little to no protein was detected in normal muscle biopsy samples. As with the mouse samples, acid activation of the lysates was required to detect expression. For TGF-β2 and 3, protein expression by ELISA was not detected. To determine the location of TGF-β1, immunofluorescence was performed on ALS and control muscle biopsy specimens (FIG. 14B, upper two rows). A pattern similar to mouse ALS muscle, with immunoreactivity identified in numerous mononuclear cells adjacent to myofiber borders, and only scant staining in control sections was observed. A second human muscle sample from a patient with end-stage ALS revealed a cluster of labeled cells in an area of grouped atrophic fibers (lowest row).

TGF-βs Induce Smads1,5 and 8 in C2C12 Muscle Cells

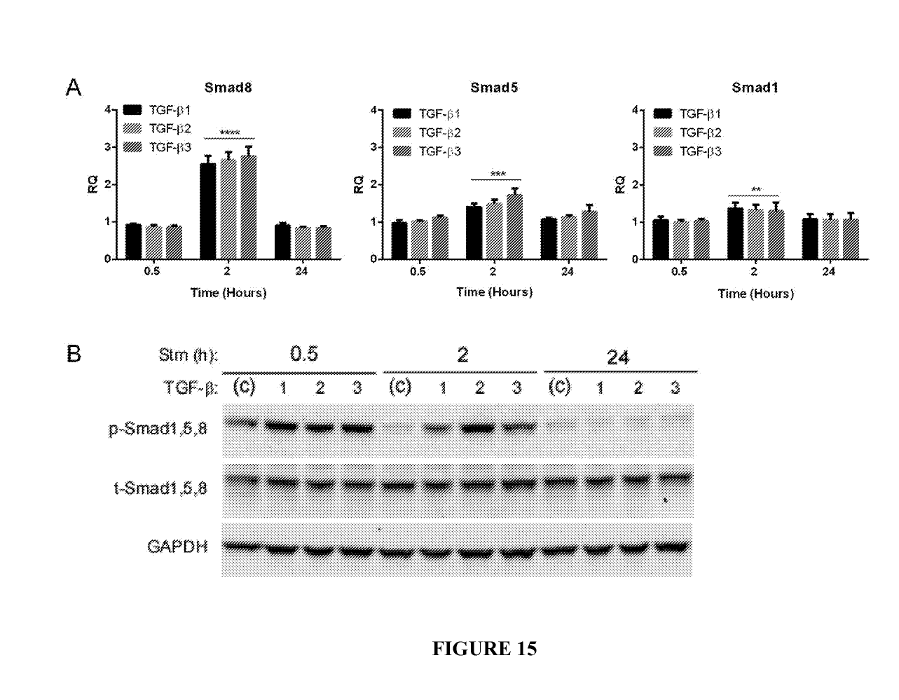

To determine a potential link with Smad1, 5 and 8 induction and activation, C2C12 muscle cells were stimulated with TGF-β ligands at different time intervals and Smad mRNA and protein (FIG. 15) was assessed. A transient and significant induction of mRNA was observed with all three isoforms, most prominently with Smad8, at two hours post stimulation. Smad8 showed a nearly 3-fold increase versus a more modest 1.3 to 1.5-fold increase with the Smad1 and 5. Interestingly, this differential pattern of expression is similar to what was observed in mouse and human

ALS muscle samples where Smad8 was significantly higher than the other two mRNAs. Phosphorylated (p)-Smad1, 5, 8 which is the activated form (FIG. 15B) was also studied. By 0.5 h there was marked induction of p-Smad1, 5, 8 equally by all three TGF-f3 isoforms which persisted through 2 h but dissipated by 24 h. Total Smad1, 5, 8 did not change with TGF-β stimulation in contrast to the increase that was observed in the ALS mouse tissue. In summary, TGF-β stimulation of muscle cells in culture recapitulated some of the patterns observed with the Smads in human and mouse ALS muscle.

### Smad2 is Increased in ALS Muscle

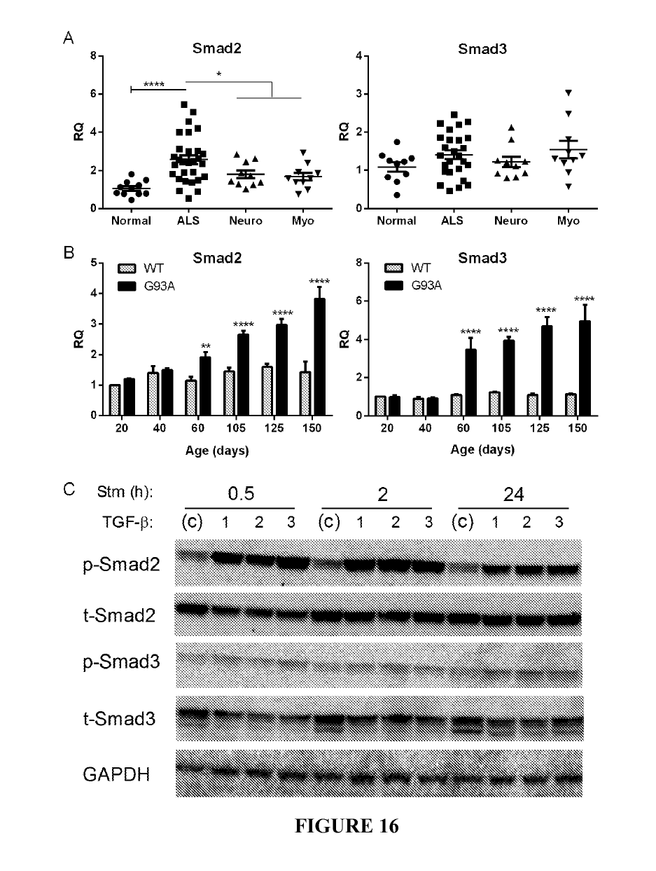

Although RNA sequencing analysis did not show significant elevation of Smads2 and 3 over disease controls, they are more typically linked with TGF-β for activation. Muscle biopsy samples were assessed by qRT-PCR and a significant, albeit a much smaller fold-increase (<2-fold) in Smad2 mRNA in ALS versus disease controls (FIG. 16A) was observed. Smad3, on the other hand, was not elevated compared to disease or normal control specimens. In the G93A mouse both targets were elevated beginning at 60 d post-natal and progressively increased toward end-stage (FIG. 16B). In C2C12 muscle cells, all three TGF-βisoforms robustly induced p-Smad2 at 0.5 and 2 h which persisted at 24 h (FIG. 16C). Smad3 on the other hand showed a more modest activation at the different time intervals. Neither Smad2 nor Smad3 mRNA was induced with TGF-β stimulation.

These studies show that TGF-β1, 2, and 3 are significantly increased in human and mouse ALS muscle and parallel the Smads. The correlation with muscle strength in the human specimens coupled with an increase in expression with clinical advancement in the ALS mouse show that these ligands are markers of disease progression. They can also be used in diagnosis or clinical staging.

A number of embodiments have been described. Nevertheless, it will be understood that various modifications can be made. Accordingly, other embodiments are within the scope of the following claims.

1-16. (canceled)

17. A method for diagnosing amyotrophic lateral sclerosis (ALS) in a subject comprising:
a) isolating a sample from the subject; and
b) detecting the level of SMAD1, SMAD2, SMAD5, SMAD8, TGFβ1, TGFβ2, TGFβ3 and/or BMP4 in the sample, an increase in SMAD1, SMAD2, SMAD5, SMAD8, TGFβ1, TGFβ2, TGFβ3 and/or BMP4, as compared to a control, indicating the subject has or is at risk for developing ALS.

18. The method of claim 17, wherein the sample is muscle tissue or blood from the subject.

19. The method of claim 17, wherein the level of mRNA encoding SMAD1, SMAD2, SMAD5, SMAD8, TGFβ1, TGFβ2, TGFβ3 and/or BMP4 is detected.

20. The method of claim 17, wherein the level of SMAD1, SMAD2, SMAD5, SMAD8, TGFβ1, TGFβ2, TGFβ3 and/or BMP4 protein is detected.

21. A method of treating a subject with or at risk of developing ALS comprising the steps of claim 17 and further comprising administering an effective amount of an agent that decreases the level of SMAD1, SMAD2, SMAD5, SMAD8, TGFβ1, TGFβ2, TGFβ3 and/or BMP4 in the subject.

22. A method for determining the efficacy of a selected treatment for ALS in a subject comprising:
a) isolating a sample from the subject before the selected treatment;
b) detecting the level of SMAD1, SMAD2, SMAD5, SMAD8, TGFβ1, TGFβ2, TGFβ3 and/or BMP4 in the sample;
c) treating the subject with the selected treatment;
d) isolating a sample from the subject after the selected treatment;
e) detecting the level of SMAD1, SMAD2, SMAD5, SMAD8, TGFβ1, TGFβ2, TGFβ3 and/or BMP4 in the sample of step d);
f) comparing the level of SMAD1, SMAD2, SMAD5, SMAD8, TGFβ1, TGFβ2, TGFβ3 and/or BMP4 detected in step b) and e), a decrease in the level of SMAD1, SMAD2, SMAD5, SMAD8, TGFβ1, TGFβ2, TGFβ3 and/or BMP4 detected in step e) indicating that the selected treatment is effective for treating ALS in the subject.

23. A method of optimizing treatment of a subject with ALS comprising the steps of claim 22 and further comprising repeating the selected treatment of the subject if the selected treatment is indicated to be effective for treating ALS in the subject or providing a different treatment to the subject is the selected treatment is not indicated to be effective for treating ALS in the subject.

24. A method for determining the progression of ALS in a subject comprising:
a) isolating a first sample from the subject with ALS;
b) detecting the level of SMAD1, SMAD2, SMAD5, SMAD8, TGFβ1, TGFβ2, TGFβ3 and/or BMP4 in the sample;
c) isolating a second sample from the subject at a later time point;
d) detecting the level of SMAD1, SMAD2, SMAD5, SMAD8, TGFβ1, TGFβ2, TGFβ3 and/or BMP4 in the sample of step d);
e) comparing the level of SMAD1, SMAD2, SMAD5, SMAD8, TGFβ1, TGFβ2, TGFβ3 and/or BMP4 detected in step b) and d), an increase in the level of SMAD1, SMAD2, SMAD5, SMAD8, TGFβ1, TGFβ2, TGFβ3 and/or BMP4 detected in step d) indicating that ALS has progressed in the subject; a decrease in the level of SMAD1, SMAD2, SMAD5, SMAD8, TGFβ1, TGFβ2, TGFβ3 and/or BMP4 detected in step d) indicating that ALS has improved in the subject; and no difference between the level of SMAD1, SMAD2, SMAD5, SMAD8, TGFβ1, TGFβ2, TGFβ3 and/or BMP4 detected in step b) and d) indicating that ALS has not progressed in the subject.

25. The method of claim 24, wherein the level of mRNA encoding SMAD1, SMAD2, SMAD5, SMAD8, TGFβ1, TGFβ2, TGFβ3 and/or BMP4 is detected.

26. The method of claim 24, wherein the level of SMAD1, SMAD2, SMAD5, SMAD8, TGFβ1, TGFβ2, TGFβ3 and/or BMP4 protein is detected.
